(19) United States
(12) Patent Application Publication
(10) Pub. No.: US 2025/0345334 A1
(43) Pub. Date: Nov. 13, 2025

(54) METHODS OF TREATING PROSTATE CANCER

(71) Applicants: The University of Chicago, Chicago, IL (US); The Board of Trustees of the University of Illinois, Urbana, IL (US)

(72) Inventors: Russell Szmulewitz, Chicago, IL (US); Anthony Williams, Chicago, IL (US); Donald Vander Griend, Oak Park, IL (US)

(21) Appl. No.: 18/657,378

(22) Filed: May 7, 2024

(51) Int. Cl. A61K 31/519; A61K 31/4166; A61K 31/4745; A61K 31/569; A61P 35/00

(52) U.S. Cl. CPC A61K 31/519; A61K 31/4166; A61K 31/4745; A61K 31/569; A61P 35/00

(57) ABSTRACT

This disclosure relates to methods for treatment of prostate cancer.

### BACKGROUND OF DISCLOSURE

### Field of Invention

This disclosure relates to a method for treatment of a subject with prostate cancer. The method comprises administering a therapeutically effective amount of a WEE1 inhibitor or a pharmaceutically acceptable salt thereof and a therapeutically effective amount of a nuclear hormone receptor signaling inhibitor (NHRSI) or a pharmaceutically acceptable salt thereof.

### Technical Background

Prostate cancer (PC) is among the most prevalent and lethal cancers in America and the second leading cause of cancer-related death among men, with an estimated 288,300 newly diagnosed cases and 34,700 deaths projected by the end of 2023 [1]. PC is characterized by the abnormal proliferation and invasion of androgen receptor (AR)-positive/prostate specific antigen (PSA)-positive luminal-secretory cells. The majority of prostate cancers express AR and depend on AR-mediated signaling for their continued survival and growth; this provides the rationale for why androgen deprivation therapy (ADT) continues to represent the standard of care for metastatic prostate cancer since it inhibits proliferation and activates the apoptotic death of these malignant cells. Although initially effective, PC progression following ADT is inevitable, marking the critical transition to the lethal form of the disease, the development of castration-resistant prostate cancer (CRPC). Unfortunately, the current therapeutic armamentarium, even highly specific and potent AR signaling inhibition (ARSI) therapies, offer only palliative care. Most men with metastatic CRPC are likely to die from their disease. A variety of mechanisms for PC tumors to overcome ARSI's have been described, including intratumoral androgen production [2], and somatic mutations/alternative splicing in the AR gene [3], and increased expression and activity of another nuclear hormone receptor, the glucocorticoid receptor (GR), which can compensate for or bypass AR to drive a proliferative transcriptional profile [4], [5]. Alternatively, ARSI resistance can occur via AR/nuclear hormone receptor-independent signaling pathways all together [6].

Concerning the latter of these mechanisms, sex determining region Y-box 2 (SOX2) has emerged in recent years as an AR-regulated transcription factor whose upregulation is associated with aggressive PC [7]. Interestingly, accumulating evidence suggests SOX2 functions as an oncogene in PC cells via a significantly different mechanism than its defined canonical function in human embryonic stem cell maintenance, likely due to the aberrant expression of known embryonic stem cell binding partners of SOX2 in PC cells [7]. SOX2 has been reported on extensively in several malignancies [8]-[11], and has been demonstrated to drive a wide spectrum of pro-oncogenic mechanisms in PC, from involvement in cancer metabolomics that promote PC cell survival [12], to lineage plasticity shifts towards AR-independent PC progression [13]. Importantly, constitutive overexpression of SOX2 in a hormone sensitive, SOX2-negative PC cell line is sufficient to sustain proliferation in the absence of AR ligand in vitro, and establish tumors in the castrate setting in vivo [7].

In the present study, we set out to define the mechanistic underpinnings for SOX2-mediated ARSI-resistance in PC. Furthermore, we sought to investigate whether SOX2 expression was sufficient to obviate AR-bypass signaling through GR. The central hypothesis of this work was that SOX2 regulates a distinct transcriptional profile, independent of nuclear hormone receptor signaling that drives therapeutic resistance. Therefore, this work seeks to uncover the underlying SOX2-mediated signaling node in metastatic CRPC, yielding data that could lead to novel targeted approaches aimed at restoring sensitivity to nuclear hormone receptor signaling inhibition, against both AR and GR.

### SUMMARY OF THE DISCLOSURE

This disclosure describes compositions and methods for treatment of a subject with prostate cancer. The method comprises administering a therapeutically effective amount of a WEE1 inhibitor or a pharmaceutically acceptable salt thereof and a therapeutically effective amount of a nuclear hormone receptor signaling inhibitor (NHRSI) or a pharmaceutically acceptable salt thereof.

In a first aspect, the present disclosure provides a method of treating prostate cancer in a subject. The method includes administering to the subject a therapeutically effective amount of (i) a WEE1 inhibitor or a pharmaceutically acceptable salt thereof; and (ii) a nuclear hormone receptor signaling inhibitor (NHRSI) or a pharmaceutically acceptable salt thereof.

In one embodiment of the first aspect, the prostate cancer is SOX2-positive.

In one embodiment of the first aspect, the prostate cancer is advanced prostate cancer. In one embodiment of the first aspect, the prostate cancer is a metastatic prostate cancer.

In one embodiment of the first aspect, the prostate cancer is a castration-resistant prostate cancer.

In one embodiment of the first aspect, the prostate cancer is a neuroendocrine prostate cancer.

In one embodiment of the first aspect, the WEE1 inhibitor is MK-1775.

In one embodiment of the first aspect, the NHRSI is enzalutamide (ENZ) and/or relacorilant.

In one embodiment of the first aspect, the NHRSI is an androgen receptor signaling inhibitor and/or a selective glucocorticoid receptor-modulating inhibitor. In one embodiment, the androgen receptor signaling inhibitor is MDV3100, ARN-509, flutamide, bicalutamide, nilutamide, apalutamide, enzalutamide, AZD3514, darolutamide, or cyproterone acetate. In one embodiment, the androgen receptor signaling inhibitor is enzalutamide. In one embodiment, the selective glucocorticoid receptor-modulating inhibitor is mifepristone or relacorilant (CORT134). In one embodiment, the selective glucocorticoid receptor-modulating inhibitor is relacorilant (CORT134).

In one embodiment of the first aspect, the WEE1 inhibitor and nuclear hormone receptor signaling inhibitor are administered simultaneously or sequentially.

In one embodiment of the first aspect, the WEE1 inhibitor and/or nuclear hormone receptor signaling inhibitor are administered orally, intravenously, subcutaneously, or intratumorally.

In a second aspect, the present disclosure provides a method of treating a subject having prostate cancer. The method includes a) selecting a subject having prostate cancer, wherein the prostate cancer exhibits resistance to treatment with a nuclear hormone receptor signaling inhibitor; and b) administering to the selected subject (i) one or more WEE1 inhibitors or a pharmaceutically acceptable salt thereof, and (ii) a nuclear hormone receptor signaling inhibitor (NHRSI) or a pharmaceutically acceptable salt thereof.

In one embodiment of the first aspect, the prostate cancer is SOX2-positive.

In one embodiment of any of the preceding aspects or embodiments thereof, the administering reduces tumor growth, invasiveness, progression, recurrence, and/or metastasis of the SOX2-positive prostate cancer in the subject.

In one embodiment of any of the preceding aspects or embodiments thereof, the method decreases proliferation in SOX2-positive prostate cancer cells, and re-sensitizes prostate cancer cells to treatment with an androgen receptor signaling inhibitor (ARSI) and a selective glucocorticoid receptor modulating (SGRM) therapy to nuclear hormone receptor signaling inhibition.

In a third aspect, the present disclosure provides a method of treating prostate cancer in a subject including the steps of a) administering to the subject a therapeutically effective amount of a composition comprising (i) one or more WEE1 inhibitors or a pharmaceutically acceptable salt thereof, and (ii) nuclear hormone receptor signaling inhibitor (NHRSI) or a pharmaceutically acceptable salt thereof (NHRSI), wherein the composition is preferentially targeted to SOX2-positive prostate cancer cells; b) decreasing proliferation in SOX2-positive prostate cancer; c) re-sensitizing prostate cancer to treatment with an androgen receptor signaling inhibitor (ARSI) and/or a selective glucocorticoid receptor modulating-inhibitor; and d) reducing growth, invasiveness, progression, recurrence, and/or metastasis of the SOX2-positive prostate cancer in the subject.

In a fourth aspect, the present disclosure provides a method of treating prostate cancer that includes the steps of a) measuring SOX2 protein expression in prostate cancer tissue obtained from a subject; and b) comparing the SOX2 protein expression of in the prostate cancer tissue with SOX2 protein expression of a non-cancerous or normal control sample, wherein decreased levels of SOX2 protein in the prostate cancer tissue indicate that the subject is sensitive to treatment with a (i) WEE1 inhibitor or a pharmaceutically acceptable salt thereof; and/or (ii) a nuclear hormone receptor signaling inhibitor (NHRSI) or a pharmaceutically acceptable salt thereof.

In one embodiment of the fourth aspect, SOX2 is a biomarker for the diagnosis, prognosis, monitoring and/or screening of or as a therapeutic or target for a prostate cancer in a subject.

In one embodiment of the fourth aspect, the expression of SOX2 in a prostate cancer cell results in an increase in expression of WEE1, E2F1, and CDK1, and desensitizes prostate cancer cells to an androgen receptor signaling inhibition and selective glucocorticoid receptor-modulating inhibition.

In one embodiment of the fourth aspect, treatment with a WEE1 inhibitor or a pharmaceutically acceptable salt thereof and a nuclear hormone receptor signaling inhibitor (NHRSI) or a pharmaceutically acceptable salt thereof reduces expression of CDK1, reduces phosphorylation of CDK1, and re-sensitizes prostate cancer cells to NHRSI therapy.

In one embodiment of the fourth aspect or any embodiments thereof, a decreased transcriptional and/or translational expression level of SOX2, when compared to transcriptional and/or translational expression level of SOX2 measured prior to treatment of the prostate cancer, is indicative of re-sensitization of SOX2-positive prostate cancer cells to NHRSI treatments and reversal of an aggressive SOX2-mediated prostate cancer progression.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure and are incorporated in and constitute a part of this specification. The drawings illustrate one or more embodiment(s) of the disclosure, and together with the description serve to explain the principles and operation of the disclosure.

FIGS. 1A-1D. RNA-seq in Control (Ctl) and -SOX2Ko Cells. Ctl or SOX2^{KO} cells were cultured in 10% charcoal-stripped media with vehicle (R-1881) or ENZ for 72 hrs, then total RNA was recovered for RNA-seq. (FIG. 1A) Venn diagrams demonstrating overlapping or unique, differentially expressed gene profiles for Ctl and SOX2^{KO} cells treated with ENZ relative to vehicle. (FIG. 1B) Top gene set pathways enriched by KEGG using Web Gestalt for unique, differentially expressed genes in SOX2^{KO} cells following ENZ treatment relative to vehicle. Pathways are ranked by strength of significance (P-value). (FIG. 1C) Normalized counts from RNA-seq of representative cell cycle-related genes (CDK1, E2F1, WEE1, CDKN2A, TGFB1, and CDKN1C) across treatment conditions for Ctl and SOX2^{KO} cells are graphed. Error bars for normalized count data represent +/−SEM for triplicate samples in each condition. Asterisks represent significant differences with the following adjusted p-values: **1E-03 to 1E-09, ***1.0E-10 to 1.0E-19, ****greater than 1.0E-20. (FIG. 1D) Western blots in Ctl and SOX2^{KO} cells treated with (3.5 μM R-1881) vehicle or ENZ (10 PM) for SOX2 and cell cycle proteins encoded by highlighted genes: WEE1, E2F1, CDK1, and p57, confirm relative expression levels observed by RNA-seq. j-Actin was blotted as a loading control.

FIGS. 2A-2C. WEE1 and CDK1 are differentially expressed genes in SOX2-HI and -LOW PC patient tumor tissue. (FIG. 2A) Distribution of TPM (transcripts per million) values for dichotomized “HI” upper quartile (dark gray; N=78) and “LOW” lower quartile (light gray; N=78) SOX2 gene expression in PC metastases (N=310) from a collection of 7 publicly available RNA-Seq datasets (left). Box plots displaying TPM differential expression of transcript levels for WEE1(FIG. 2B) and CDK1 (FIG. 2C) in patient samples with SOX2-HI quartile (dark gray) vs -LOW quartile (light gray) gene expression. Error bars for TPM and box plot data represent +/−SEM of all samples in HI and LOW quartiles. Asterisks represent p<0.0001.

FIGS. 3A-3C. SOX2-mediated GR regulation and in vitro sensitivity to SGRM treatment. (FIG. 3A) SOX2 ChIP qPCR demonstrating significant enrichment of SOX2 chromatin binding at the NR3C1 promoter (charcoal), relative to IgG (light gray); Western blotting shows positive regulation of GR in Ctl cells relative to SOX2Ko cells; IGV tracks from SOX2 ChIP-seq indicate a strong DNA binding peak (charcoal) at exon 2 of the NR3C1 gene. (FIG. 3B) Western blotting on Ctl vs SOX2^{KO} cells grown in 10% CSS supplemented with R-1881 and treated with (left to right) ENZ (AR-inhibited), ENZ/DEX (AR-inhibited/GR-active), and ENZ/DEX/CORT134 (AR-inhibited/GR-active/GR-inhibited) over 3 days for SOX2, GR, AR, and SGK1 (canonical gene target of GR). 3-Actin was blotted as a loading control. (FIG. 3C) Proliferation curves over 6 days for Ctl and SOX2Ko cells grown in 10% CSS supplemented with R-1881 (R) and treated with (left to right): vehicle, R (circle), ENZ (square), DEX (triangle), ENZ/DEX (diamond), and ENZ/DEX/CORT134 (X). Error bars for ChIP-qPCR data represent +/−SEM for 3 technical replicates tested in IgG- and SOX2-immunoprecipitated DNA. Error bars for proliferation data represent +/−SEM of 6 replicates tested in each condition. AU=Arbitrary Units measuring intensity of fluorescence.

FIGS. 4A-4D. WEE1 inhibition re-sensitizes SOX2-positive PC cells to NHRSI treatment. Western blotting in control (Ctl) cells cultured in 10% CSS supplemented with R-1881 and treated with (left to right): Vehicle (R), AR-inhibited (R/ENZ), and AR-inhibited/GR-inhibited conditions (R/ENZ/DEX/CORT134)+/−WEE1 inhibitor (FIG. 4A) MK1775 and WEE1, CDK1, and pCDK1 (indicator of canonical WEE1 activity). β-Actin was blotted as a loading control. Proliferation curves for AR-inhibited (square) and WEE-inhibited (X) Ctl cells alone compared to AR inhibition combined with WEE1 inhibition treatment (asterisk) using MK1775 (FIG. 4B). Proliferation curves over 6 days for SGRM-treated (triangle) and WEE1-inhibited(X) Ctl cells compared to SGRM treatment combined with WEE1 inhibition (asterisk) using MK1775 (FIG. 4C). All conditions were compared to vehicle (blue), supplemented with R-1881. Error bars for proliferation curves represent +/−SEM of 6 replicates tested in each condition. AU=Arbitrary Units measuring intensity of fluorescence. (FIG. 4D) Kaplan-Meier curves comparing mice treated with AR inhibition combined with WEE1i inhibition (charcoal) and vehicle treatment (light grey).

FIGS. 5A-5C. WEE1 inhibition induces mitotic catastrophe in SOX2-positive PC cells. (FIG. 5A) Western blotting in CWRR1-Ctl cells cultured in 10% CSS supplemented with R-1881 and treated with: Vehicle (R; 3.5 μM R-1881), and AR-inhibited conditions (3.5 pM R/10 μM ENZ)+/−0.8 μM MK-1775 (WEE1 inhibition) comparing phosphorylated Histone H3 (indicator of cells in M phase) and cleaved PARP (indicator of apoptosis). 3-Actin was blotted as a loading control. (FIG. 5B) Propidium Iodide staining of CWRR1-Ctl cells treated with either 10 μM ENZ (ARSI alone) or 10 μM ENZ in combination with 0.8 μM MK-1775 (ARSI-/WEE1i-treated) for 6 days. % cells at G2/M cell cycle phase increased from 11.95% (SEM+/−0.64) following ENZ treatment (charcoal bar), compared to 21.1% (SEM+/−1.56) following ENZ+WEE1 inhibition (light gray bar). (FIG. 5C) β-galactosidase activity in CWRR1-Ctl cells treated with either 10 μM ENZ or 10 μM ENZ in combination with 0.8 μM MK-1775 for 6 days. Fold change relative to vehicle (3.5 μM R-1881) was unchanged for cells treated with ENZ (0.99-fold change; charcoal bar), but was significantly increased in cells treated with ENZ+WEE1i (1.3-fold change; light gray bar). Error bars for propidium iodide staining and β-galactosidase activity data represent +/−SEM of 6 replicates tested in each condition, respectively.

FIGS. 6A-6B. WEE1-targeted therapy inhibits proliferation of SOX2-positive neuroendocrine PC. (FIG. 6A) Western blotting in NCI-H660 cells cultured in 5% CSS supplemented with 100 nM DEX (GR agonism) and treated with: 0.8 μM MK-1775 (WEE1 inhibition), 1 μM CORT134 (GR modulation), or 0.8 μM MK-1775 with 1 μM CORT134 (WEE1 inhibition/GR modulation) comparing SOX2, AR, GR, CDK1, phosphorylated CDK1 and phosphorylated Histone H3. Treatment with 10 nM hydrocortisone (HC) was used as a control for DEX supplementation and cell viability. j-Actin was blotted as a loading control. (FIG. 6B) CellTiter Blue assay measuring % cell viability after treatment of NCI-H660 cells in 100 nM DEX (charcoal bar), 1 μM CORT134 (gray bar) or 1 μM CORT134 with 0.8 μM MK-1775 (light gray bar) for 6 days. Relative to l0 nM HC treatment, cell viability was unchanged for DEX- and CORT134-treated NCI-H660 cells (104.8%, SEM+/−0.030 and 91.8%, SEM+/−0.041, respectively), but was significantly reduced in CORT134+MK-1775-treated cells (28.1% SEM+/−0.021). Error bars for cell viability data represent +/−SEM of 6 replicates tested in each condition.

FIG. 7. Schematic representation of hypothesis for WEE1 inhibition to increase sensitivity to NHRSI in PC. SOX2 expression in PC allows bypass of NHRSI's alone via mediation of cell cycle progression through the WEE1-CDK1 signaling axis, leading to treatment resistance and disease progression. However, the addition of WEE1 inhibition to NHRSI treatment prevents maintenance of genetic instability in SOX2-positive PC by dephosphorylation of CDK1 which allows PC cells to progress through the G2/M checkpoint, inducing replicative stress and eventually mitotic catastrophe, resulting in restoration of NHRSI sensitivity and tumor regression.

### DETAILED DESCRIPTION

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

All publications, patents and patent applications cited herein are hereby expressly incorporated by reference in their entirety for all purposes.

### Definitions

Before describing the methods and compositions of the disclosure in detail, a number of terms will be defined. As used herein, the singular forms “a.” “an.” and “the” include plural referents unless the context clearly dictates otherwise. For example, reference to “a therapeutic target” means one or more therapeutic targets.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

In some embodiments, percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

The term “cancer” as used herein refers to an abnormal growth of cells which proliferate in an uncontrolled way and, in some cases, to metastasize (spread).

The term “prostate cancer,” as used herein, refers to both primary prostate tumors as well as metastases of primary prostate tumors. Prostate cancer can be histologically or cytologically confirmed as an adenocarcinoma of the prostate and/or small cell/neuroendocrine cancer of the prostate.

As used herein, the terms “advanced prostate cancer,” “locally advanced prostate cancer,” “advanced disease,” and “locally advanced disease” refer to prostate cancers that have extended through the prostate capsule, and are meant to include stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced disease, and these patients have substantially less favorable outcomes compared to patients having clinically localized (organ-confined) prostate cancer. Locally advanced disease is clinically identified by palpable evidence of induration beyond the lateral border of the prostate, or asymmetry or induration above the prostate base. Locally advanced prostate cancer can be diagnosed pathologically following radical prostatectomy if the tumor invades or penetrates the prostatic capsule, extends into the surgical margin, or invades the seminal vesicles.

As used herein, the terms “metastatic prostate cancer” and “metastatic disease” refer to prostate cancers that have spread to regional lymph nodes or to distant sites, and are meant to include stage D disease under the AUA system and stage T×N×M+under the TNM system. As is the case with locally advanced prostate cancer, surgery is generally not indicated for patients with metastatic disease, and hormonal (androgen ablation) therapy is the preferred treatment modality.

“Pharmaceutical composition” as used herein refers to a composition that includes one or more therapeutic agents disclosed herein, such as a WEE1 inhibitor, a nuclear hormone receptor signaling inhibitor (NHRSI), a pharmaceutically acceptable salt, carrier, a solvent, an adjuvant, and/or a diluent, or any combination thereof.

As used herein, the terms “therapeutic amount,” “therapeutically effective amount,” or “effective amount” can be used interchangeably and refer an amount of a compound that becomes available through an appropriate route of administration to provide a therapeutic benefit to a patient for a disorder, a condition, or a disease. The amount of a compound which constitutes a “therapeutic amount,” “therapeutically effective amount,” or “effective amount” will vary depending on the compound, the disorder and its severity, and the age of the subject to be treated, but can be determined routinely by one of ordinary skill in the art.

For example, an effective amount of a WEE1 inhibitor, such as MK-1775 and/or PD0166285; for example, an effective amount of a nuclear hormone receptor signaling inhibitor (NHRSI), such as enzalutamide (ENZ), and/or relacorilant (also referred to herein as CORT125134 or CORT134); or a combination of a WEE1 inhibitor and an NHRSI includes an amount sufficient to alleviate the signs, symptoms, or causes of prostate cancer. As another example, an effective amount of a WEE1 inhibitor, such as MK-1775 and/or PDO166285; an NHRSI, such as enzalutamide (ENZ) and/or relacorilant; or a combination of a WEE1 inhibitor and an NHRSI includes an amount sufficient to alleviate the signs, symptoms, or causes of metastatic or castration-resistant prostate cancer. As another example, an effective amount of a WEE1 inhibitor, such as MK-1775; an NHRSI, such as enzalutamide (ENZ) and/or relacorilant (CORT134); or a combination of a WEE1 inhibitor and an NHRSI includes an amount sufficient to prevent the development of prostate cancer.

Thus, a therapeutically effective amount can be an amount that slows, reverses, or prevents tumor growth or advancement, increases survival time, or inhibits tumor progression or metastasis. Also, for example, an effective amount of a WEE1 inhibitor, such as MK-1775 and/or PD0166285; an NHRSI, such as enzalutamide (ENZ) and/or relacorilant; or a combination of a WEE1 inhibitor and an NHRSI includes an amount sufficient to cause a therapeutic or clinical improvement in a subject having prostate cancer when administered to the subject. The effective mount can vary with the stage of the prostate cancer being treated, the type and concentration of one or more compositions (e.g., comprising a WEE1 inhibitor, such as MK-1775; an NHRSI, such as enzalutamide (ENZ) and/or relacorilant (CORT134); or a combination of a WEE1 inhibitor and an NHRSI) administered, and the amounts of other drugs that are also administered.

A therapeutically effective amount can be determined by such considerations as may be known in the art. The amount must be effective to achieve the desired therapeutic effect in a subject suffering from prostate cancer. The therapeutically effective amount depends, inter alia, on the type and severity of the disease to be treated and the treatment regimen. The therapeutically effective amount is typically determined in appropriately designed clinical trials (e.g., dose range studies) and the person versed in the art will know how to properly conduct such trials in order to determine the therapeutically effective amount. As generally known, a therapeutically effective amount depends on a variety of factors including the distribution profile of a therapeutic agent (e.g., WEE1 inhibitor, such as MK-1775; an NHRSI, such as enzalutamide (ENZ) and/or relacorilant (CORT134); or a combination of a WEE1 inhibitor and an NHRSI) or composition within the body, the relationship between a variety of pharmacological parameters (e.g., half-life in the body) and undesired side effects, and other factors such as age and sex, etc.

“Treating” or “treatment,” as used herein, covers the treatment of a disorder, condition, or a disease described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, ameliorating, or slowing progression of one or more symptoms of the disease or disorder. For example, the terms “treating,” “treat,” or “treatment” refer to either preventing development or exacerbation of, providing symptomatic relief for, or curing a patient's disorder, condition, or disease.

For purposes of this invention, treating prostate cancer includes, without limitation, alleviating one or more clinical indications, decreasing tumor growth or tumor cell proliferation, reducing the severity of one or more clinical indications of prostate cancer condition, diminishing the extent of the condition, stabilizing the subject's disease state (i.e., not worsening), delay or slowing, halting, or reversing cancer progression, and bringing about partial or complete remission. Treating prostate cancer also includes prolonging survival by days, weeks, months, or years as compared to prognosis if treated according to standard medical practice not incorporating or administering a WEE1 inhibitor or a nuclear hormone receptor signaling inhibitor according to a method provided herein.

As used herein, the terms “administering” and “administration” include oral administration, topical contact, administration as a suppository, intravenous, intraperitoneal, intramuscular, intralesional, intratumoral, intrathecal, intranasal (e.g., inhalation, nasal mist or drops), or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering a therapeutically effective amount of a WEE1 inhibitor, such as MK-1775 and/or PD0166285; an NHRSI, such as enzalutamide (ENZ), and/or relacorilant; or a combination of a WEE1 inhibitor and a NHRSI according to methods of the present invention for preventing or relieving one or more symptoms associated with prostate cancer.

As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. The two or more structurally different compounds can be comprised of a WEE1 inhibitor (e.g., MK-1775 and/or PD0166285) and an NHRSI (e.g., enzalutamide (ENZ), and/or relacorilant (CORT134). In some embodiments, the co-administered compounds are administered by the same route. In other embodiments, the co-administered compounds are administered via different routes. For example, one compound can be administered orally, and the other compound can be administered, e.g., sequentially or simultaneously, via intravenous, intramuscular, subcutaneous, or intraperitoneal injection. The simultaneously or sequentially administered compounds or compositions can be administered such that a WEE1 inhibitor and an NHRSI are simultaneously present in a subject or in a cell at an effective concentration

As used herein, the terms “patient,” “subject,” and “individual” can be used interchangeably and refer to an animal. For example, the patient, subject, or individual can be a mammal, such as a human to be treated for a disorder, condition, or a disease.

It is noted that terms like “preferably,” “commonly,” and “typically” are not utilized herein to limit the scope of the methods and compositions as described herein or to imply that certain features are critical, essential, or even important to the structure or function of the subject matter recited in the claims.

As used herein, the term “level of expression” or “expression level” refers to a measurable level of expression of the products of biomarkers, such as, without limitation, the level of messenger RNA transcript expressed or of a specific exon or other portion of a transcript, the level of proteins or portions thereof expressed of the biomarkers, the number or presence of DNA polymorphisms of the biomarkers, the enzymatic or other activities of the biomarkers, and the level of specific metabolites.

As used herein, the term “differentially expressed” or “differential expression” refers to a difference in the level of expression of the biomarkers that can be assayed by measuring the level of expression of the products of the biomarkers, such as the difference in level of messenger RNA transcript or a portion thereof expressed or of proteins expressed of the biomarkers. In a preferred embodiment, the difference is statistically significant. The term “difference in the level of expression” refers to an increase or decrease in the measurable expression level of a given biomarker, for example, as measured by the amount of messenger RNA transcript and/or the amount of protein in a sample as compared with the measurable expression level of a given biomarker in a control.

As used herein, the term “control” or “control sample” refers to a specific value or dataset that can be used to prognose or classify the value, e.g. expression level or reference expression profile obtained from the test sample associated with an outcome class. A person skilled in the art will appreciate that the comparison between the expression of the biomarkers in the test sample and the expression of the biomarkers in the control will depend on the control used. For example, in various aspects described herein, the control is determined from biological sample(s) from healthy individuals/populations.

As used herein, “sample” or “biological sample” is referred to in its broadest sense, and includes solid and liquid or any biological sample obtained from nature, including an individual, body fluid, cell line, tissue culture, or any other source, which may contain genetic material. As indicated, biological samples include body fluids, such as blood, semen, lymph, sera, plasma, urine, synovial fluid, spinal fluid, sputum, pus, sweat, as well as liquid samples from the environment such as plant extracts, pond water and so on. Solid samples may include animal or plant body parts, including but not limited to hair, fingernail, leaves and so on.

As used herein, the term “WEE1” refers to a nuclear serine/threonine kinase encoded by the WEE1 gene in humans. WEE1 is also known as “WEE1 G2 checkpoint kinase” and “WEE1 kinase”. WEE1 activates cell cycle checkpoints by phosphorylating and thus inhibiting cyclin and CDK activity. WEE1 functions in regulation of the G2 M checkpoint, the cell size checkpoint, and the DNA damage checkpoint. In higher eukaryotes, WEE1 is inactivated by phosphorylation and degradation. The SCF protein complex (an E3 ubiquitin ligase) regulates WEE1 by ubiquitination. Additionally, recognition of WEE1 by SCF is mediated by phosphorylation of WEE1 by Polio-like kinase 1 (PLK1) and CDC2. WEE1 is also negatively regulated by Kruppel-like factor 2 (KLF2).

The terms “WEE1 inhibitor” or “WEE1i” refers to any compound (e.g., a pharmaceutically active compound) that reduces or eliminates WEE1 activity. WEE1 inhibitors, for example, can result in the reduction or elimination of WEE1 activation by one or more signaling molecules, proteins, or other compounds, or can result in the reduction or elimination of WEE1 activation by all signaling molecules, proteins, or other compounds. The term also includes compounds that decrease or eliminate the activation or deactivation of one or more proteins or cell signaling components by WEE1 (e.g., a WEE1 inhibitor can decrease or eliminate WEE1-dependent inactivation of cyclin and CDK activity). WEE1 inhibitors also include compounds that inhibit WEE1 expression (e.g., compounds that inhibit WEE1 transcription or translation). Examples of WEE1 inhibitors include, but are not limited to MK-1775 and/or PD0166285.

As used herein, the terms “nuclear hormone receptor signaling inhibitor,” “NHRSI,” or “nuclear hormone receptor signaling inhibition therapy” refers to any compound that reduces or eliminates the expression and activity of nuclear hormone receptors, such as the androgren receptor (AR), AR-mediated signaling, or glucocorticoid receptor (GR). NHRSI inhibitors, for example, can result in the reduction or elimination of nuclear hormone receptor signaling that drives drug resistance in prostate cancer by one or more signaling molecules, proteins, or other compounds, or can result in the reduction or elimination of nuclear hormone receptor signaling that drives drug resistance in prostate cancer by all signaling molecules, proteins, or other compounds. NHRSI inhibitors include, but are not limited to, androgen receptor signaling inhibitors and/or a selective glucocorticoid receptor-modulating inhibitors.

As used herein, the terms “androgen receptor (AR) signaling inhibitor”, “ARSI”, “AR antagonist” or “AR inhibitor” refers to any compound that inhibits or reduces at least one activity of an androgen receptor polypeptide. Exemplary AR activities include, but are not limited to, co-activator binding, DNA binding, ligand binding, or nuclear translocation. Examplary AR inhitors include MDV3100, ARN-509, flutamide, bicalutamide, nilutamide, apalutamide, enzalutamide, AZD3514, darolutamide, or cyproterone acetate.

As used herein, the terms “selective glucocorticoid receptor-modulating inhibitor” “selective GR modulating (SGRM) therapy” or “glucocorticoid receptor antagonist” refers any compound which inhibits any biological response associated with the binding of GR to an agonist. GR modulators include, but are not limited to, mifepristone or relacorilant (CORT134).

As used herein, an “antagonist” refers to an antagonist which, at an effective concentration, essentially completely inhibits an activity of an AR polypeptide or capable of partially inhibiting an activity of an AR polypeptide. By “essentially completely” is meant at least about 80%, at least about 90%, at least about 95%, at least about 96%, at least about 97%, at least about 98% at least about 99%, or greater inhibition of the activity of an AR polypeptide.

As used herein, the terms “sex determination Y regional transcription factor 2,” “sex determining Region Y-box2,” or “SOX2,” refers to AR-regulated transcription factor whose upregulation is associated with aggressive prostate cancer. SOX2 functions as an oncogene in PC cells via a significantly different mechanism than its defined canonical function in human embryonic stem cell maintenance, likely due to the aberrant expression of known embryonic stem cell binding partners of SOX2 in prostate cancer cells [7]. SOX2 has been reported on extensively in several malignancies [8]-[11], and has been demonstrated to drive a wide spectrum of pro-oncogenic mechanisms in PC, from involvement in cancer metabolomics that promote PC cell survival [12], to lineage plasticity shifts towards AR-independent PC progression [13]. Importantly, constitutive overexpression of SOX2 in a hormone sensitive, SOX2-negative PC cell line is sufficient to sustain proliferation in the absence of AR ligand in vitro, and establish tumors in the castrate setting in vivo [7]. Furthermore, the presence of SOX2 (SOX2 positive) in prostate cancer cells serves as a biomarker to predict insensitivity to nuclear hormone receptor signaling inhibition, as well as indicate the use of a WEE1-targeted therapy to improve patient management in SOX2-positive prostate cancer.

As used herein, “biomarker” refers to a characteristic that can be objectively measured that indicates the normal and/or pathogenic biological processes or pharmacological response of one or more cells. For example, in one embodiment, the biomarkers are molecules, genes or proteins known in the art to be associated with a biological activity such as loss of pluripotency, cell proliferation, apoptosis, cytotoxicity or differentiation. In one embodiment, the biomarkers are detected using immunodetection with antibodies. In one embodiment, the step of detecting an effect of a compound comprises detecting the expression of one or more biomarkers. By way of example, biomarkers contemplated herein can include, but are not limited to, SOX2, WEE1, E2F1, CDK1, and TGFβ.

As used herein, the term “combination therapy” refers to the administration of at least two pharmaceutical compounds to a subject to treat prostate cancer. The two compounds may be administered simultaneously, or sequentially in any order during the entire or portions of the treatment period. The two compounds may be administered following the same or different dosing regimens. In some cases, one compound is administered following a scheduled regimen while the other compound is administered intermittently. In some cases, both compounds are administered intermittently.

### Overview

ARSI's are a central component in the clinical management of advanced PC across multiple disease states [57]. However, selective pressure applied by ARSI therapy portends treatment failure and acquired drug resistance, at least in part through the upregulation and selection of PC cells expressing key oncogenic drivers, including SOX2 and GR. Understanding how these resistance mechanisms may interact and can be targeted is of paramount importance. We previously demonstrated that constitutive overexpression of SOX2 in a hormone-sensitive, SOX2-negative cell line is sufficient to drive tumor growth in castrated mice [7]. In the present study, we extended our previous findings to demonstrate that SOX2 silencing in a castration-resistant, endogenously SOX2 expressing PC cell line effectively restores ARSI treatment sensitivity. This finding stands in contrast to SOX2-positive PC cells, which have unchanged proliferative capacity following AR pathway modulating agonism or antagonism.

We also provide evidence herein that NR3C1 (the gene encoding GR) is a positively-regulated SOX2 target gene in PC. To our knowledge, this is the first report to describe direct regulation of GR expression by SOX2 in PC. Despite SOX2's positive regulation of NR3C1, canonical GR target gene expression was sustained following GR activation with agonist ligand in SOX2^{KO} cells. Interestingly, GR agonism or selective modulation/antagonism only impacted PC cell proliferation in the SOX2^{KO} context, and had no effect on SOX2-positive PC cells. This was a particularly important finding, as these data indicate that SOX2 expression de-sensitizes PC cells to nuclear hormone receptor manipulation more broadly, where SOX2-positive PC cells are unaffected by AR or GR agonism, and drive an aggressive, resistant phenotype despite ARSI and GR modulation treatment.

To uncover the underlying molecular mechanism driving SOX2-mediated indifference to nuclear hormone receptor modulation, we utilized transcriptional profiling of SOX2-positive and -negative cells following nuclear hormone receptor antagonism, which demonstrated strong enrichment of key genes in pathways predominantly related to cell cycle control. Specifically, we identified WEE1 and CDK1, two differentially expressed genes in a SOX2-dependent context, which have well-defined roles in cell cycle regulation and progression in cancer. WEE1 is a protein kinase that acts as a tumor suppressor in non-malignant, eukaryotic cells [29]. In response to DNA damage, WEE1 phosphorylates and inactivates CDK1, thereby inhibiting the CDK1-Cyclin B complex from downstream activities that drive cell cycle progression through the G2/M checkpoint [29]. Conversely, WEE1 kinase acts as an oncogene in several malignancies by helping cells to maintain a tolerable level of genetic instability [28], [29]. In line with these findings, our data reveal significantly decreased levels of WEE1 and CDK1 gene and protein expression in SOX2-negative PC cells after NHRSI, and significantly higher expression of WEE1 and CDK1 in CRPC patient tumors with high SOX2 expression. Additionally, in line with the described mechanism of action for WEE1i, our findings indicate an accumulation of PC cells at the G2/M checkpoint as well as protein expression of biomarkers consistent with caspase-mediated cell death and cellular senescence activity following treatment with MK-1775.

WEE1i has emerged as a promising approach to improve cancer patient management, and is currently being investigated in several malignancies [44]. Critically, use of WEE1 inhibitor MK-1775 in combination with NHRSI's was sufficient to overcome treatment insensitivity of NHRSI's administered alone in SOX2-positive PC, both in vitro and in vivo. Moreover, this effect was observed in both adenocarcinoma and neuroendocrine PC models.

Our study has several strengths in support of its findings and conclusions. It used NHRSI targeting two distinct nuclear hormone receptors, AR and GR, to demonstrate that SOX2 imparts resistance to the class, and not just to ARSI. Additionally, the correlation between WEE1, CDK1, and SOX2 gene expression in metastatic CRPC patient tissue we report herein strengthens the translational significance of these findings. Furthermore, the SOX2-positive, NHRSI-resistant phenotype (reversible by WEE1 inhibition) was exhibited in both adenocarcinoma- and neuroendocrine-derived cell lines, CWRR1 and NCI-H660, respectively. The demonstration of our phenotype in NCI-H660 cells suggests the potential use of WEE1 inhibition to improve patient response in the aggressive, neuroendocrine PC setting, where current treatment outcomes are poor.

Several reports suggest that the efficacy of WEE1i treatment is dependent on an altered p53 status [29], [38], [45], and importantly both of our model systems tested for these studies have altered p53 status; CWRR1 cells harbor an A273H mutation of exon 8 [46], and NCI-H660 cells harbor an exon 9-11 deletion [46]. It is believed that, due to the gatekeeping function of p53 at the G0/G1 interphase and S phase, inactivation of p53 in malignant cells causes an increased dependence of cell cycle regulation on the G2/M checkpoint. Presumably, this underlies the historic focus to date on the ability of WEE1i to improve the efficacy of DNA damaging agents [41], DNA damage repair-targeted drugs [42], and radiotherapy [47], [48], modalities for which the success of the therapeutic intervention is limited due to loss or inactivity of p53, as well as toxicity of other radiosensitizing agents [48]. Further mechanistic studies on the potential interdependence of our findings with respect to WEE1, SOX2, and genomic alterations of p53 are warranted.

Expression of SOX2 in PC has been reported to drive lineage plasticity in a p53/Rb-deficient PC model that describes a shift from epithelial to an aggressive, neuroendocrine-like phenotype in response to ARSI challenge [13]. Data from our RNA-Seq did not prioritize pathways consistent with this finding, nor did we identify differential expression of genes encoding proteins such as Synaptophysin or Chromogranin A, commonly associated with the neuroendocrine PC phenotype. However, given the reported success of WEE1i in p53-deficient malignancies, and our findings linking WEE1i sensitivity to SOX2-expressing NHRSI-resistant PC cells, WEE1i alone or in combination with NHRSI should be explored as a therapeutic option for SOX2-positive, p53/Rb-deficient PC patients with a specifically aggressive, ARSI-resistant phenotype.

In addition to cell cycle regulation, our RNA-Seq data following NHRSI treatment prioritized pathways associated with dysregulation of DNA damage repair. This observation is in line with the known role of WEE1 in response to DNA damage [29], and could suggest an opportunity to improve the efficacy of drugs targeting DNA damage response pathways through the use of a WEE1 inhibitor. TGFB1 encodes the protein TGFβ, a cytokine with wide-ranging physiologic and immune regulatory functions [33]. TGFβis reported to have both tumor suppressive and tumor promoting function, where in the former it transcriptionally and post-translationally regulates several cell cycle inhibitors and cyclin-CDK complexes to induce cell cycle arrest [41]. Tumor cells have the ability to escape the growth suppressive function of TGFβin different malignancies, mediated by major oncogenes such as c-Myc and Ras [32], [33]. Our data are rationally consistent with these findings, as TGFB1 was significantly upregulated upon SOX2 silencing in PC cells subsequent to ARSI and TGFβsignaling was prioritized as a key pathway associated with differential gene expression in NHRSI-treated PC cells +/−SOX2 by our RNA-seq. SUMOylation was another prioritized pathway enriched in a SOX2-dependent context following NHRSI treatment. SUMOylation describes a reversible, post-translational modification critically involved in several molecular regulatory mechanisms, including DNA damage repair, as well as immune response and cell cycle progression [49]. The extent to which SUMOylation is regulated by SOX2, or contributes to SOX2-mediated NHRSI treatment resistance in PC, warrants further investigation. The identification of these other important pathways further highlights the need for in-depth investigation of potential therapeutic targets for SOX2-positive PC.

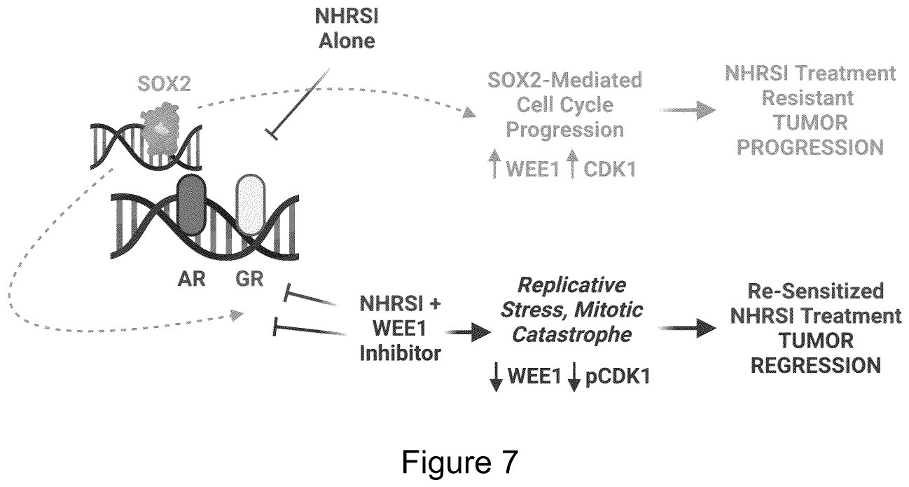

New strategies to extend the benefit of NHRSI, or overcome NHRSI resistance in PC, are in high demand. Herein, we present compelling evidence to implicate SOX2 as a mediator of NHRSI insensitivity in PC via cell cycle dysregulation. Although SOX2 has traditionally proven difficult to directly target pharmacologically [50], this work represents an opportunity to leverage SOX2 expression as a bona fide biomarker that predicts insensitivity to NHRSI therapy, as well as simultaneously suggest the use of a targeted therapy against WEE1 to drive NHRSI-resistant PC cells into replicative stress and mitotic catastrophe, thus improving NHRSI efficacy (FIG. 7). The use of WEE1i to counter SOX2-mediated NHRSI resistance is a promising therapeutic option for patients that are likely to suffer from poor treatment outcomes, and future efforts will focus on translating our findings clinically.

### Methods

In a first aspect, the present disclosure provides a method of treating prostate cancer in a subject. The method includes administering to the subject a therapeutically effective amount of (i) a WEE1 inhibitor or a pharmaceutically acceptable salt thereof; and (ii) a nuclear hormone receptor signaling inhibitor (NHRSI) or a pharmaceutically acceptable salt thereof.

In one embodiment of the first aspect, the prostate cancer is SOX2-positive.

In one embodiment of the first aspect, the prostate cancer is advanced prostate cancer. In one embodiment of the first aspect, the prostate cancer is a metastatic prostate cancer.

In one embodiment of the first aspect, the prostate cancer is a castration-resistant prostate cancer.

In one embodiment of the first aspect, the prostate cancer is a neuroendocrine prostate cancer.

In one embodiment of the first aspect, the WEE1 inhibitor is MK-1775.

In one embodiment of the first aspect, the NHRSI is enzalutamide (ENZ) and/or relacorilant.

In one embodiment of the first aspect, the NHRSI is an androgen receptor signaling inhibitor and/or a selective glucocorticoid receptor-modulating inhibitor. In one embodiment, the androgen receptor signaling inhibitor is MDV3100, ARN-509, flutamide, bicalutamide, nilutamide, apalutamide, enzalutamide, AZD3514, darolutamide, or cyproterone acetate. In one embodiment, the androgen receptor signaling inhibitor is enzalutamide. In one embodiment, the selective glucocorticoid receptor-modulating inhibitor is mifepristone or relacorilant (CORT134). In one embodiment, the selective glucocorticoid receptor-modulating inhibitor is relacorilant (CORT134).

In one embodiment of the first aspect, the WEE1 inhibitor and nuclear hormone receptor signaling inhibitor are administered simultaneously or sequentially.

In one embodiment of the first aspect, the WEE1 inhibitor and/or nuclear hormone receptor signaling inhibitor are administered orally, intravenously, subcutaneously, or intratumorally.

In a second aspect, the present disclosure provides a method of treating a subject having prostate cancer. The method includes a) selecting a subject having prostate cancer, wherein the prostate cancer exhibits resistance to treatment with a nuclear hormone receptor signaling inhibitor; and b) administering to the selected subject (i) one or more WEE1 inhibitors or a pharmaceutically acceptable salt thereof, and (ii) a nuclear hormone receptor signaling inhibitor (NHRSI) or a pharmaceutically acceptable salt thereof.

In one embodiment of the first aspect, the prostate cancer is SOX2-positive.

In one embodiment of any of the preceding aspects or embodiments thereof, the administering reduces tumor growth, invasiveness, progression, recurrence, and/or metastasis of the SOX2-positive prostate cancer in the subject.

In one embodiment of any of the preceding aspects or embodiments thereof, the method decreases proliferation in SOX2-positive prostate cancer cells, and re-sensitizes prostate cancer cells to treatment with an androgen receptor signaling inhibitor (ARSI) and a selective glucocorticoid receptor modulating (SGRM) therapy to nuclear hormone receptor signaling inhibition.

In a third aspect, the present disclosure provides a method of treating prostate cancer in a subject including the steps of a) administering to the subject a therapeutically effective amount of a composition comprising (i) one or more WEE1 inhibitors or a pharmaceutically acceptable salt thereof, and (ii) nuclear hormone receptor signaling inhibitor (NHRSI) or a pharmaceutically acceptable salt thereof (NHRSI), wherein the composition is preferentially targeted to SOX2-positive prostate cancer cells; b) decreasing proliferation in SOX2-positive prostate cancer; c) re-sensitizing prostate cancer to treatment with an androgen receptor signaling inhibitor (ARSI) and/or a selective glucocorticoid receptor modulating-inhibitor; and d) reducing growth, invasiveness, progression, recurrence, and/or metastasis of the SOX2-positive prostate cancer in the subject.

In a fourth aspect, the present disclosure provides a method of treating prostate cancer that includes the steps of a) measuring SOX2 protein expression in prostate cancer tissue obtained from a subject; and b) comparing the SOX2 protein expression of in the prostate cancer tissue with SOX2 protein expression of a non-cancerous or normal control sample, wherein decreased levels of SOX2 protein in the prostate cancer tissue indicate that the subject is sensitive to treatment with a (i) WEE1 inhibitor or a pharmaceutically acceptable salt thereof; and/or (ii) a nuclear hormone receptor signaling inhibitor (NHRSI) or a pharmaceutically acceptable salt thereof.

In one embodiment of the fourth aspect, SOX2 is a biomarker for the diagnosis, prognosis, monitoring and/or screening of or as a therapeutic or target for a prostate cancer in a subject.

In one embodiment of the fourth aspect, the expression of SOX2 in a prostate cancer cell results in an increase in expression of WEE1, E2F1, and CDK1, and desensitizes prostate cancer cells to an androgen receptor signaling inhibition and selective glucocorticoid receptor-modulating inhibition.

In one embodiment of the fourth aspect, treatment with a WEE1 inhibitor or a pharmaceutically acceptable salt thereof and a nuclear hormone receptor signaling inhibitor (NHRSI) or a pharmaceutically acceptable salt thereof reduces expression of CDK1, reduces phosphorylation of CDK1, and re-sensitizes prostate cancer cells to NHRSI therapy.

In one embodiment of the fourth aspect or any embodiments thereof, a decreased transcriptional and/or translational expression level of SOX2, when compared to transcriptional and/or translational expression level of SOX2 measured prior to treatment of the prostate cancer, is indicative of re-sensitization of SOX2-positive prostate cancer cells to NHRSI treatments and reversal of an aggressive SOX2-mediated prostate cancer progression.

Provided herein are therapeutic compositions and methods for treating prostate cancer in a subject.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Loss of SOX2 Expression Results in Differentially Expressed Genes and Specifically Enriched Pathways Associated with Resistance to AR Antagonism

### Introduction

Previously, it was reported that SOX2 expression increases with AR pathway inhibition and increased expression is associated with more aggressive disease [7]. Furthermore, constitutive overexpression of SOX2 in hormone-sensitive, SOX2-negative LAPC4 PC cells confers ability to grow tumors in vivo under castrate conditions [7]. The previous report [12]was confirmed that endogenous SOX2 expression in CWRR1 cells confers resistance to AR antagonism and silencing of SOX2 expression is sufficient to sensitize the same cell line to AR antagonism.

Based on these findings, the mechanisms underlying the association between SOX2 expression and resistance to AR pathway inhibition were investigated.

### Materials and Methods

Cell Culture: The CWR22-R1 (CWRR1) castration-resistant prostate cancer cell line was maintained in RPMI growth media containing 2 mM L-Glutamine (Gibco-Thermo Fisher Scientific, Waltham MA) supplemented with 1% penicillin/streptomycin (Corning, Corning NY) and 10% Fetal Bovine Serum (FBS; Atlanta Biologicals, Flower Branch GA). At 80-90% confluency, cells were passaged at a ratio of 1:8 into fresh media and new flasks until ready for downstream experiments. The HEK293T human embryonic kidney cell line was maintained in DMEM growth media (Gibco-Thermo Fisher Scientific, Waltham MA) supplemented with 1% penicillin/streptomycin (Corning, Corning NY) and 10% Fetal Bovine Serum (FBS; Atlanta Biologicals, Flower Branch GA). At 70-80% confluency, cells were passaged at a ratio of 1:10 into fresh media and new flasks until ready for downstream experiments. All cell lines were grown in a humidified incubator at 37° C. with 5% COa.Cell lines and subsequent genetically modified cell lines used were authenticated by the Genetics Core Facility at the University of Arizona and screened for mycoplasma contamination using the Universal Mycoplasma Detection Kit per manufacturer's specifications (American Type Tissue Culture, Manassas VA) prior to and following all experiments conducted.

Western Blotting: Following specified drug treatments for nuclear hormone receptor agonism, antagonism and selective modulation, whole cell lysates were prepared in RIPA buffer (1% Triton X-100, 50 mM Tris-HCl, 150 mM NaCl, 1% Sodium Dexoxycholate, 0.1% SDS, and 1 mM EDTA). Whole cell lysates were sonicated in 1.5 mL Eppendorf tubes using a wand that delivered 2 watts of voltage in 10 second bursts with 10 seconds of rest for 3 cycles. The protein concentration in each sample was estimated using the BCA Protein Assay Kit (Pierce-Thermo Fisher Scientific, Waltham MA) following manufacturer specifications. Lysates were boiled in Laemmli buffer (Biorad, Hercules CA) supplemented with 10% β-Mercaptoethanol for 5 min at 95° C., and cooled to room temperature. 75 g of total protein was resolved by SDS-PAGE at 100V followed by wet transfer onto PVDF membranes (LI-COR Biosciences, Lincoln NE). Non-specific interactions of antibodies with PVDF membrane-bound proteins were blocked by incubation in 1× TBS+5% dry milk (Better Living Brands, Pleasanton CA) for 1 h at room temperature. Immunoblotting was performed with primary antibodies against the following proteins of interest at respective concentrations, diluted in 1× TBS+5% dry milk at 4° C. overnight: rabbit anti-SOX2, 1:500 (D6D9; Cell Signaling Technologies, Danvers MA), rabbit anti-β2F1, 1:1000 (Cell Signaling Technologies, Danvers MA), rabbit anti-WEE1, 1:1200 (D10D2; Cell Signaling Technologies, Danvers MA), mouse anti-CDK1, 1:500 (POH1; Cell Signaling Technologies, Danvers MA), rabbit anti-p57 Kip2, 1:500 (Cell Signaling Technologies, Danvers MA), rabbit anti-phospho-CDK1, 1:500 (Tyr15 10A11; Cell Signaling Technologies, Danvers MA), and rabbit anti-GR, 1:1000 (D8H2; Cell Signaling Technologies, Danvers MA). Mouse anti-β-Actin, 1:5000 (AC-15; Millipore-Sigma, St Louis MO) was used as a loading control. Following primary antibody incubation, blots were washed 3 times in 1× TBST for 5 min each wash, and then incubated with infrared dye (IR)-conjugated secondary antibodies diluted in 1× TBST+5% dry milk for 1 h at room temperature to visualize antibody-protein interactions. Either goat anti-rabbit IRDye 800 or goat anti-mouse IRDye 680 secondary antibodies were used, 1:10,000 (LI-COR Biosciences, Lincoln NE). Following secondary antibody incubation, blots were washed in 1× TBST for 5 min each wash, and then imaged using the LI-COR Odyssey System (LI-COR Biosciences, Lincoln NE).

CRISPR-Cas9 Gene Editing: Generation of CRISPR/Cas9-mediated SOX2 knockout cell lines (SOX2^{KO}) was conducted as previously described [12]. Limited dilution was performed to isolate three clonal knockout cell lines, and successful knockout of SOX2 protein was validated by Western blot. The SOX2^{KO} clone with the greatest silencing efficiency was used for experiments presented herein. Cells stably expressing Cas9 only with no crRNA transfection were used in parallel as a control for the SOX2-positive phenotype (SOX2-Ctl).

Proliferation Assays: 2.0×10⁶ HEK293T cells were seeded in a 10 cm culture dish in complete DMEM growth media and allowed to attach overnight. HEK-293T cells were then co-transfected with Trans Lentiviral Packaging Mix (GE Healthcare Dharmacon, Lafayette CO) and the PGK-H2BeGFP lentiviral vector, a gift from Mark Mercola [14](#21210; Addgene, Watertown MA) with Lipofectamine 2000 (Invitrogen-Thermo Fisher Scientific, Waltham MA) in low-volume OptiMEM culture media (Gibco-Thermo Scientific, Waltham MA) overnight. Transfection media was replaced with complete DMEM growth media and cells were allowed to grow for 48h. Viral media was harvested and viral particles were concentrated using LentiPac Virus Concentrator (GeneCopoeia, Rockville MD) for 24 h at 4° C. Concentrated viral media was centrifuged at 5000 g, and pelleted viral particles were resuspended in complete RPMI growth media supplemented with 5 g/ml polybrene (Millipore-Sigma, St Louis MO) and used to transduce CWRR1-Ctl and SOX2Ko cells for 48h. Concentrated viral media was then replaced with full RPMI, and transduction efficiency of stable nuclear GFP expression was evaluated by fluorescent microscopy. Ctl- and SOX2Ko-GFP cells were seeded in 96-well plates (1.5×10⁴ cells per well) and allowed to adhere overnight. Culture media was then replaced with RPMI supplemented with 10% charcoal stripped fetal bovine serum (CSS; Gemini Bio-Products, West Sacramento CA) and WEE1 inhibitors, nuclear hormone receptor agonists and antagonists, as indicated for respective experiments. Drug-treated 96-well plates were then placed in the Incucyte S3 μLive Imaging platform (Essen BioScience, Ann Arbor MI). Cell proliferation was tracked by fluorescent imaging of nuclear GFP every 4h for a total of 150h. In each condition, a total of 6 replicates were collected, and mean fluorescence intensity was averaged at each time point.

In Vitro Drug Treatment: For AR agonism, a stock solution of 10 nM R-1881 (Fisher Scientific, Hampton NE) was prepared in 100% ethanol, and diluted to 3.5 μM final concentration for use in experiments. For AR antagonism, a stock solution of 10 mM enzalutamide (ENZ) in DMSO was prepared from compound provided by Astellas Pharma Global Development, Inc/Pfizer, Inc, and diluted to 10 μM final concentration for subsequent use. For GR agonism, a stock solution of 1 mM dexamethasone (DEX; Thermo Fisher Scientific, Waltham MA) was prepared in 100% ethanol, and diluted to 100 nM final concentration for subsequent use. For GR antagonism, a stock solution of 10 mM relacorilant, referred to throughout as CORT134, was prepared in 100% ethanol from compound provided by Corcept Therapeutics, and diluted to a final concentration of 1 M for subsequent use. For WEE1 inhibition, 10 mM stock solutions of MK-1775 and PD0166285 (Selleck Chemicals, Houston TX) were prepared in DMSO, and diluted to 800 nM and 1.2 mM final concentrations, respectively. In RNA-Seq experiments, cells were treated with R-1881+ENZ for 72h, followed by 6 h treatment with DEX or DEX+CORT134 prior to total RNA recovery. Following drug treatments, cell viability was evaluated using the CellTiter-Blue Cell Viability Assay (Promega, Madison WI) according to the manufacturer's protocol. Drug treatments for immunoblotting and proliferation experiments were given for the duration of the experiment, 72 h and 150 h, respectively. Each drug treatment was controlled by treatment of samples with vehicle only, 100% ethanol or DMSO, at the volume corresponding to the concentration of drug given. For in vitro nuclear hormone receptor agonism, antagonism and drug treatments, cells were cultured in respective media supplemented with 10% charcoal-stripped fetal bovine serum (CSS; Gemini Bio-Products, West Sacramento CA).

RNA-Sequencing and Pipeline Analysis: CWRR1-Ctl or -SOX2^{KO} cells were plated in a 10 cm dish in triplicate per experimental condition. Following drug treatments as indicated, attached cells were washed twice in 1×PBS (Gibco-Thermo Fisher Scientific, Waltham MA) on ice, and total RNA was recovered from samples using the RNAeasy Mini Kit (Qiagen, Hilden GERMANY) according to the manufacturer's protocol. 20 μL at a concentration of 100 ng/l was provided to the Functional Genomics Facility at the University of Chicago for assessment of RNA integrity and RNA library preparation. Following library preparation, libraries were sequenced on the Illumina NovaSeq platform (Illumina, San Diego CA) in 100 bp, paired-end reads. FASTQ files for each sample were uploaded to Galaxy (usegalaxy.org/), an open source, web-based platform containing a suite of analytical and data processing tools for intensive biomedical research. First, Trimmomatic was used to trim low-quality and adapter sequences [15]. Trimmed reads were mapped to the human reference genome GRCh37/hg19 using Bowtie2 [16]. Feature counts were generated from mapped sequencing reads to quantify gene expression levels [17]. The DESeq2 tool was then used to assess differential expression between conditions of interest (i.e., R-1881/ENZ relative to R-1881 alone, and R-1881/ENZ/DEX/CORT134 relative to R-1881/ENZ/DEX) [18]. In addition to a results file listing statistically significant, differentially expressed genes, the DESeq2 tool also produces a normalized counts file for each replicate tested, and principal component analysis along with a sample-to-distance plot as quality control for conditions being tested. A stringency cut-off of <20% or >80% in any given principal component direction was applied to assure efficiency of technical replicates in each condition compared. Finally, the AnnotateMyIDs tool was used to assign gene symbols to Ensembl gene IDs for both normalized counts and differentially expressed gene files. Only genes differentially expressed above a 1.5-fold change (FC) (˜0.58 log₂ FC) with an adjusted p-value <0.05 relative to vehicle were considered in analyses. Venn diagrams to identify overlapping or unique, differentially expressed genes for comparison of treatment conditions were generated using Venny 2.1 (bioinfogp.cnb.csic.es/tools/venny/index.html) and modified for figure presentation. Overlapping or unique differentially expressed gene lists were then used as input for WebGestalt (www.webgestalt.org/#), a functional enrichment analysis webtool [19]. Specific gene sets from KEGG, REACTOME and WikiPathway Cancer that described significantly enriched pathways from differentially expressed gene lists were subsequently used as inputs for GSEA (www.gsea-msigdb.org/gsea/index.jsp), a computational method that determines whether an a priori defined set of genes shows statistically significant, concordant differences between two biological states [20], [21]. Normalized count lists, along with.gmx files identified through WebGestalt were used for this analysis. Heatmaps generated by GSEA were converted using Morpheus (software.broadinstitute.org/morpheus/) for clearer gene annotation. Additionally, the 271 gene set describing overlapping, differentially expressed genes following ARSI and SGRM treatment in SOX2^{KO} cells was loaded into the Ingenuity Pathway Analysis program (Qiagen, Hilden GERMANY) for further investigation of enriched pathways and key upstream and downstream effectors of proliferation and PC progression.

Use of Publicly Available RNA-Sequencing Datasets: Access to the following RNA-Seq datasets was requested and approved via NIH DbGaP. IRB approval was obtained when necessary. These dataset were: phs001141.vl.pl, phs000909.vl.pl, phs000915.v2.p2, phs000310.vl.pl, phs000985.vl.pl, phs000443.vl.pl, and phs001698.vl.pl [57]-[62]. Quality control of raw FASTQ data was determined using FastQC (www.bioinformatics.babraham.ac.uk/projects/fastqc). Sequences were trimmed using cutadapt (custom parameters: a=file:/mnt/store3/clusterilab/reference/adapters/TnuSeq_R1.fa; A=file:/mn(/store3/clusterilab/reference/adapters/TruSeq_R2.fa; q=20,20; nextseq-trim=20; m=20:20: report=minimal) [Martin M]. Quality trimming and adapter trimming was also conducted (custom parameters: min length=20:20: p=20,20: 5′ adapter =AGATCGGAAGAGCACACGTCTGAACTCCAGTCA (SEQ ID NO: 1): 5′ adapter (read2)=AGATCGGAAGAGCGTCGTGTAGGGAAAGAGTGT (SEQ ID NO: 2)). Genome alignment was performed using STAR using the hg38 human reference genome [28]. PCR duplicates were removed using Picard. Reads were cross-checked against a reference database using BWE MEM for human ribosomal sequences from NCBI and Ensembl [29]. After culling duplicate datasets and those with sub-optimal QCs and alignment, we had 663 RNA-seq datasets for further analyses. Genomic features were quantified using FeatureCounts (custom parameters: reference =hg38_mRNA.gtf; parameters=-t exon -g gene_id -p -s 2; reference=reference_flattened.saf; parameters=-p -F SAF -O --fraction -f -s 2; Ensembl annotations for human genome v.hg38) [Genecode human release 26, [17]]. Gene isoforms were quantified with Kallisto, using k-mer-based pseudo-alignment and expectation maximization to probabilistically assign reads to isoforms using the human mRNA and long-non-coding transcriptome from Ensembl, genome hg38 [30]. Normalized transcript per million (TPM) values from SOX2-HI and -LOW quartiles were used as input for GSEA (as described earlier) to assess the clinical relevance of findings reported from RNA-seq data in PC cell lines. Pairwise statistics between genes of interest using normalized TPM values was conducted using a Mann-Whitney U test.

Statistical Analyses: GraphPad Prism 9.0 (GraphPad Software, San Diego CA) was the primary platform used for statistical analyses of data. For comparison of proliferation curves generated by Incucyte Live Imaging, one-way ANOVA tests were done, with Sidak's test to correct for multiple comparisons using statistical hypothesis testing, at a family-wise alpha threshold and confidence level of 0.05. For comparison of gene expression levels relative to SOX2-HI and -LOW quartiles in clinical samples, we performed unpaired, nonparametric Mann-Whitney tests using respective FPKMs in each quartile for genes of interest. Mann-Whitney tests were conducted in a two-tailed fashion at a confidence level of 95%. IC50 calculations were made by performing nonlinear regression analysis on normalized data collected from Cell-Titer Blue assays. For RNA-Seq experiments, the DESeq2 tool used for analysis automatically generates a log₂ fold change, standard error estimate for the log₂ fold change, and an adjusted p-value for multiple testing with the Benjamini-Hochberg method which controls for false discovery rate. For statistically significant, differentially expressed genes generated by DESeq2, we converted log 2 fold changes to fold changes using the following equations: FC=−(2(^{ABS (log}^{₂}^{FC)})); for positive FC values, FC=2(^{ABS(log}^{₂}^{FC)}) was used to preserve FC directionality.

### Results

To examine the mechanisms underlying the association between SOX2 expression and resistance to AR pathway inhibition, CWRR1-Ctl and -SOX2^{KO} cells were grown for 72 h in 10% CSS supplemented with low (castrate) levels of R-1881 (3.5 pM), an AR-agonist, with or without 10 M ENZ, a potent AR-antagonist widely used to treat advanced PC [23]. After which, total RNA was recovered from each cell line to be used for preparation of RNA libraries for RNA Sequencing (RNA-Seq). Following RNA-Seq, differentially expressed gene profiles (genes with >1.5-fold difference and adjusted p-value <0.05) for cells treated with an AR inhibitor were relatively compared to vehicle under AR agonism.

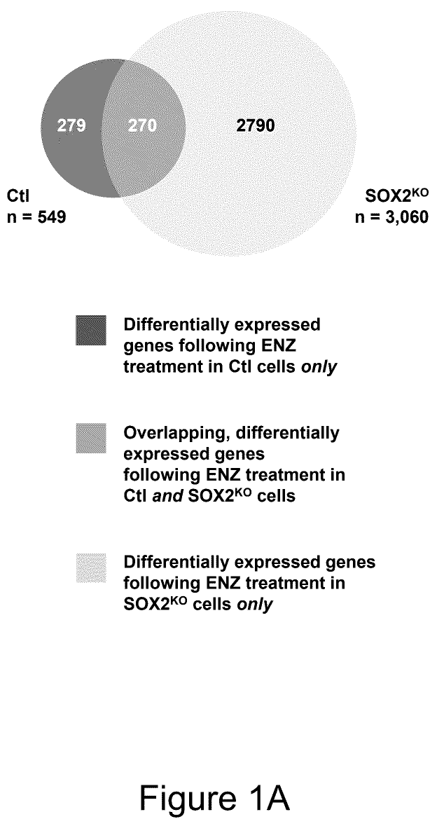

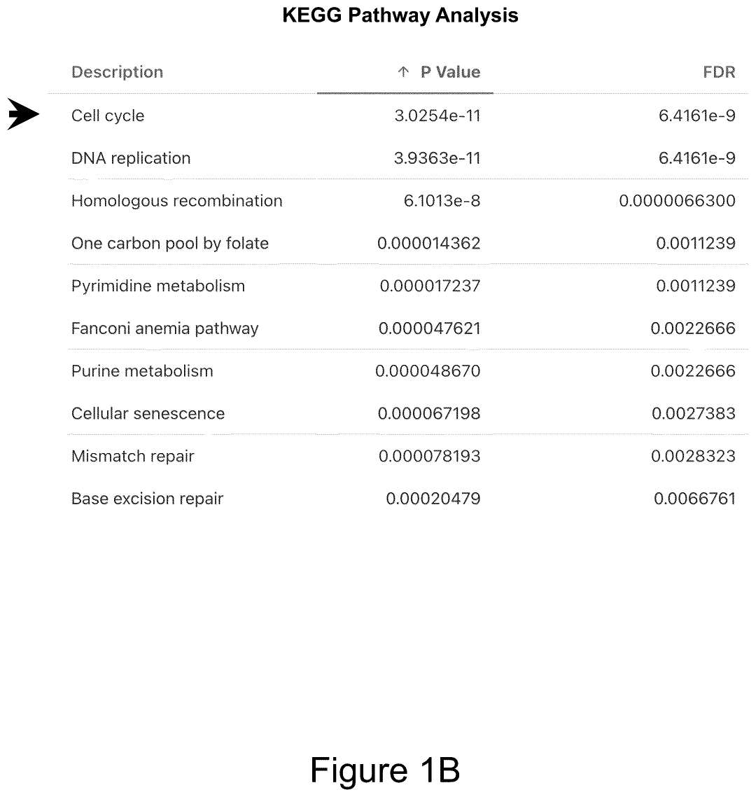

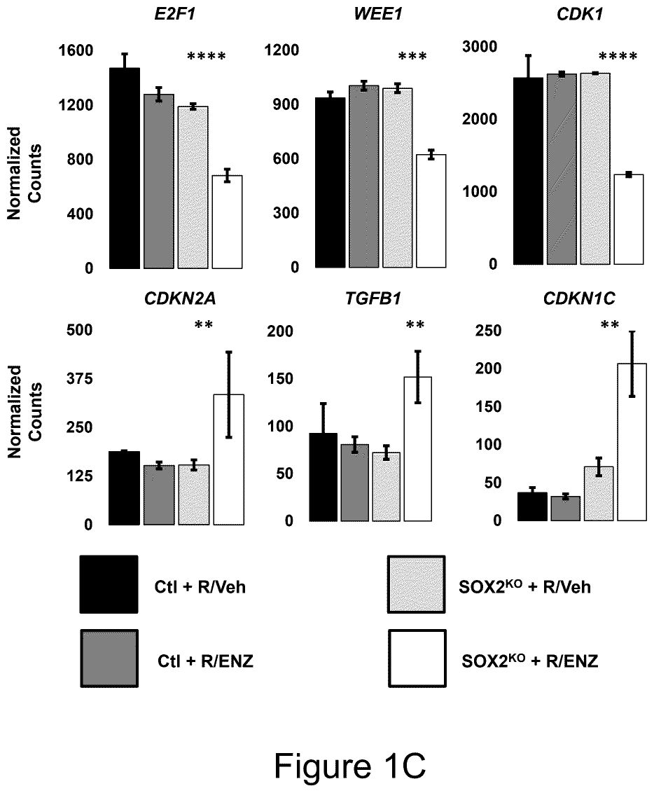

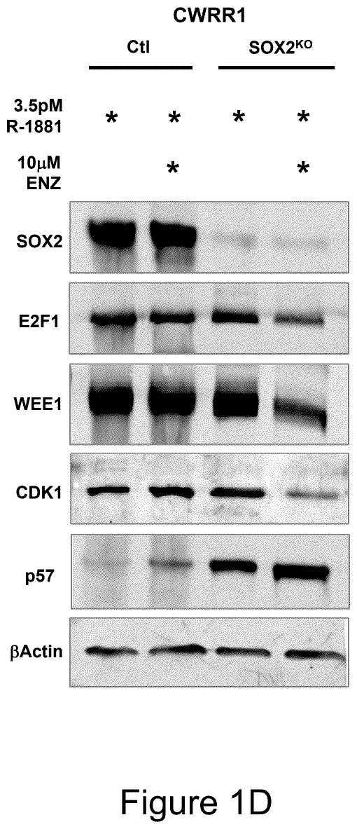

The results showed a total of 549 genes were differentially expressed in AR-inhibited Ctl cells relative to vehicle, compared to 3,040 genes differentially expressed in AR-inhibited SOX2Ko cells relative to vehicle, nearly 5 times as many genes (FIG. 1A). Of the 549 genes differentially expressed in Ctl cells, nearly half (n=270) overlap with differentially expressed genes in SOX2^{KO} cells (FIG. 1A). Interestingly, the overwhelming majority of genes differentially expressed in SOX2Ko cells (n=2770) treated with ENZ are unique to SOX2Ko cells that are not differentially expressed genes in Ctl cells (FIG. 1A). This observation led to the exploration in greater depth the unique, differentially expressed genes following AR Inhibition in SOX2Ko cells with the hypothesis that, genes or gene networks in this subset were responsible for the sensitivity to AR antagonism recovered with SOX2 loss, and conversely, that SOX2 expression may be driving ARSI-resistance through these same genes and networks. To do so, the 2770 differentially expressed genes unique to SOX2^{KO} cells were analyzed with the three distinct pathway enrichment programs: KEGG, Reactome, and WIKI Pathways Cancer. The most highly enriched pathways with a significant p-value and false discovery rate (FDR) that had overlap with at least one other pathway enrichment program were identified and further characterized. As demonstrated in FIG. 1B, which illustrates the KEGG pathway analysis, Cell Cycle was the most highly enriched pathway of the differentially expressed genes in SOX2^{KO} cells subsequent to AR inhibition. Several genes show strong differential expression in AR-inhibited SOX2^{KO} cells, in sharp contrast to AR-inhibited Ctl cells, where relative gene expression between AR inhibition and -agonism was largely not distinguishable. Of note, several well-established drivers and regulators of cell cycle progression, which exemplify this difference between Ctl and SOX2^{KO} cells are found in the KEGG Cell Cycle gene set, and include CDK1, E2F1, WEE1, CDKN2A, TGFB1 and CDKN1C. Normalized counts for CDKI, E2F1, WEE1, CDKN2A, TGFB1 and CDKN1C were plotted for both Ctl and SOX2^{KO} cells across conditions in FIG. 1C. The expression of E2F1, WEE1 and CDK1, drivers of cell cycle progression in several cancers [24]-[29], are relatively high and consistent in Ctl cells across conditions, and are similar to normalized count levels in AR-agonized SOX2^{KO} cells. However, normalized count levels for these genes decrease significantly among AR-inhibited SOX2^{KO} cells (FIG. 1C). Conversely, CDKN2A, TGFB1 and CDKN1C expression (negative regulators of cell progression [30]-[35]) remains low for both conditions in Ctl cells, and rises sharply upon AR inhibition in SOX2^{KO} cells relative to AR agonism (FIG. 1C). Finally, differential expression observed at the transcriptomic level were validated with Western blotting for protein expression analyses. Consistent with RNA-Seq, and as demonstrated in FIG. 1D, E2F1, WEE1 and CDK1 expression levels are notably lower in SOX2^{KO} cells upon AR inhibition. Conversely p57, the protein encoded by CDKN1C, has lower expression in Ctl compared to SOX2^{KO} cells, and has the highest expression with AR antagonism (FIG. 1D).

### CONCLUSIONS

Taken together, these data indicate that SOX2 expression de-sensitizes PC cells to ARSI, and that the observed phenotype following SOX2 knockout in PC cells is imparted through an inability to maintain cell cycle-related gene and protein expression programs observed in SOX2-expressing PC cells.

### Example 2: High SOX2 Expression Positively Correlates with WEE1 Expression and Negatively Correlates TGFB1 Expression in Metastatic CRPC Tissues

### Introduction

Using in vitro models in Example 1, a strong association between SOX2 expression and enrichment of key drivers and regulators of cell cycle related pathways subsequent to AR antagonism (FIGS. 1A-1D) was demonstrated. Further, a recent report performing SOX2 IHC in a tissue microarray demonstrates SOX2-positive PC tumors were significantly associated with decreased time to metastasis and PC-specific death [12]. Thus experiments were conducted in an effort to clinically validate this finding.

### Methods

A publicly available dataset produced by the SU2C Prostate Cancer East Coast Dream Team, where tissues were collected from men with metastatic CRPC (n=310) and processed for transcriptome-wide RNA-Seq [22]was used.

To assess how cell cycle related genes related to SOX2 expression in this cohort, TPM values were first dichotomized for SOX2 expression into quartiles, using the top and bottom quartiles to represent SOX2 HI (n=78) and SOX2 μLOW (n=78) expression, respectively.

### Results

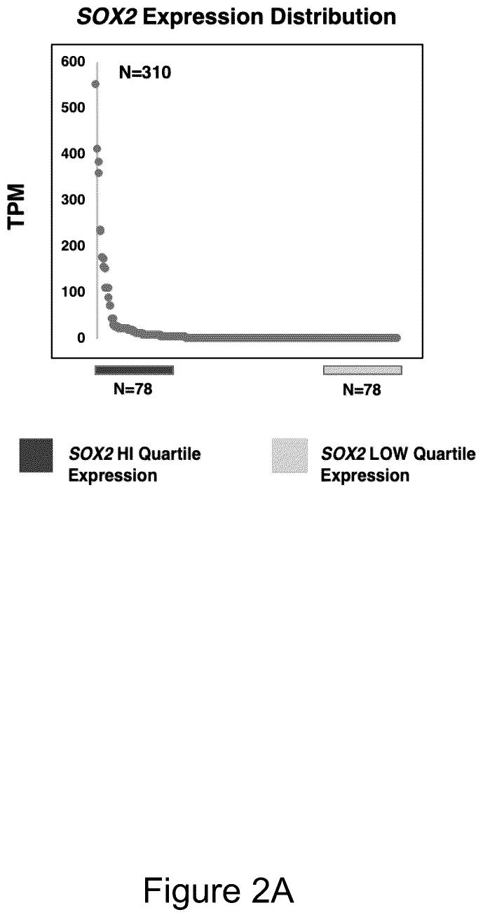

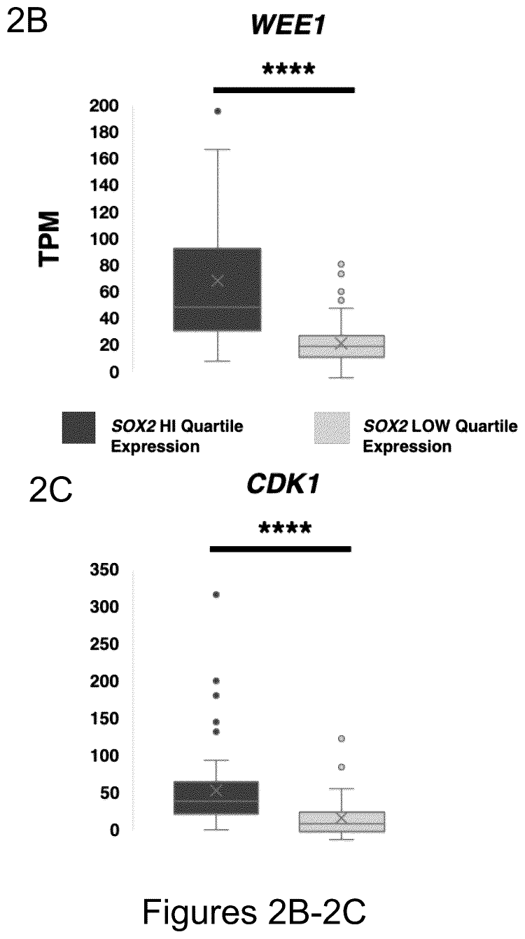

The distribution of SOX2 expression across the cohort is shown in FIG. 2A where patient samples in the SOX2 μLOW quartile are labeled green, and patient samples in the SOX2 HI quartile are labeled red. Average TPM values for SOX2 HI and SOX2 μLOW quartiles were 54.33 (SEM+/−11.55) and 0.013 (SEM+/−0.002), respectively (FIG. 2A). Next, GSEA was conducted on the SOX2 HI and SOX2 μLOW expression quartiles to test for gene correlations using the same KEGG Cell Cycle Pathway gene set employed to analyze the RNA-Seq data from the in vitro model system described earlier. Analogous to what was observed in the CWRR1 PC cell line, in clinical CRPC samples, there is a strong relationship between SOX2 expression and key cell cycle drivers and regulators; WEE1 and CDK1 expression was upregulated in the SOX2 HI quartile relative to SOX2 μLOW (FIGS. 2B-2C), at p<0.0001 for both genes. CDKN2A and E2F1 expression was also upregulated in the SOX2 HI quartile relative to SOX2 μLOW (not shown, p<0.0001), while expression differences between SOX2 HI and LOW quartiles for TGFB1 and CDKN1C did not reach statistical significance (not shown, p=0.3108 and p=0.1723, respectively). These data serve to validate our findings from in vitro studies linking SOX2 expression and sensitivity to AR-targeted therapy, using a cohort of CRPC tissues that demonstrate a corresponding relationship between HI and LOW SOX2 expression, and oncogenic drivers and regulators of the cell cycle, respectively.

### CONCLUSIONS

These data, importantly, serve to validate the findings from in vitro studies linking SOX2 expression and sensitivity to AR-targeted therapy, using a cohort of CRPC tissues that demonstrate a corresponding relationship between HI and LOW SOX2 expression, and oncogenic drivers and regulators of the cell cycle, respectively.

### Example 3: Glucocorticoid Receptor (GR) is a Positively Regulated SOX2 Target Gene in Prostate Cancer, and Sensitivity to GR Pathway Modulation is Dependent on SOX2 Expression

### Introduction

GR is a nuclear hormone receptor that shares DNA response element homology with AR, and is associated with resistance to AR pathway inhibition [4]. As SOX2 is similarly associated with resistance to AR signaling inhibition (ARSI) and demonstrated that loss of SOX2 restores ARSI sensitivity, experiments were conducted to test whether there was a relationship between SOX2, GR expression, and GR-mediated signaling. The SOX2 cistrome in PC was recently reported [12].

### Results

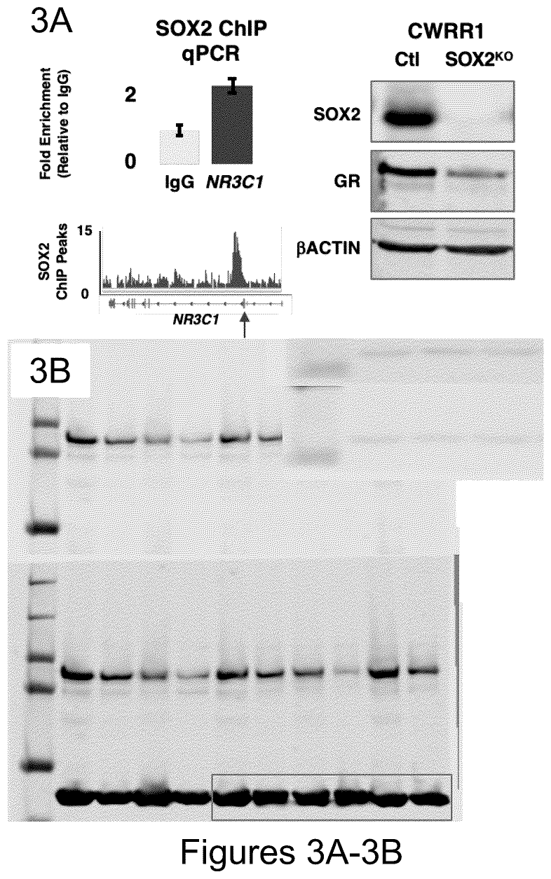

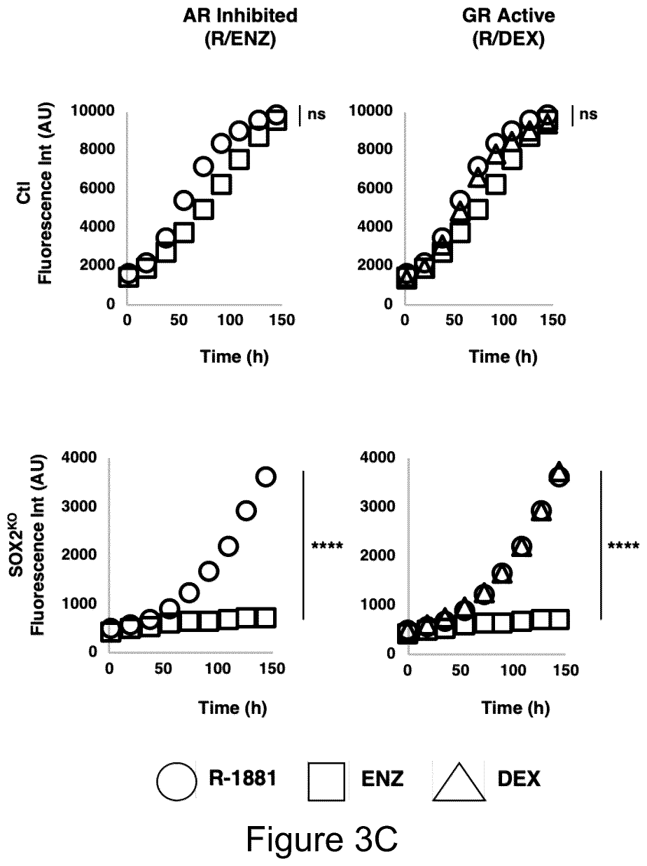

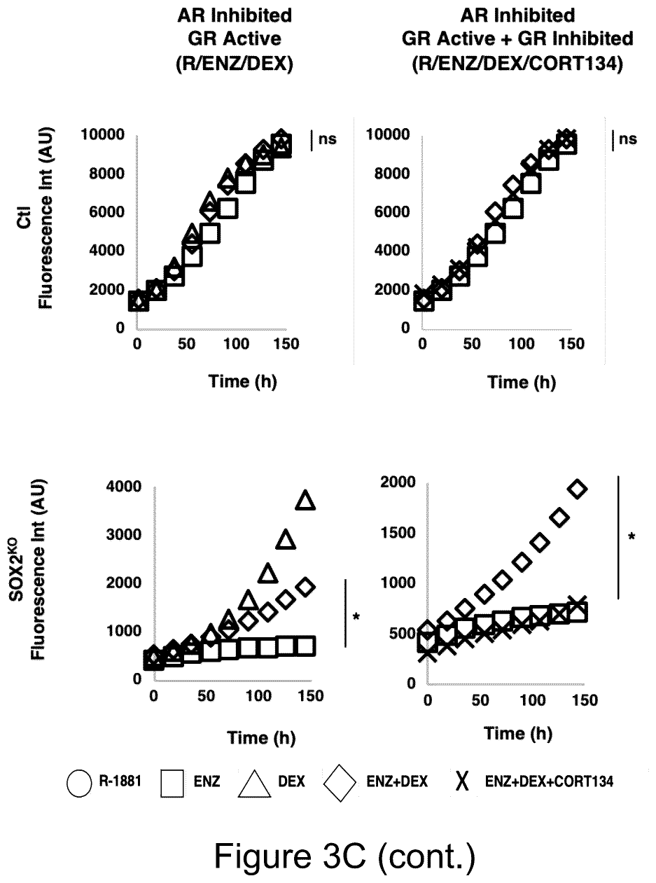

Based on analysis of these data, which suggests the presence of a SOX2 binding site in the NR3C1 gene promoter (gene that encodes the GR protein), SOX2 ChIP-qPCR confirms SOX2 engagement of the NR3C1 promoter within 200 bp of the transcription start site (TSS) in CWRR1 PC cells, as well as a strong SOX2-bound DNA peak near the NR3C1 TSS of exon 2 using Integrative Genome Viewer (IGV) (FIG. 3A). Promoter engagement at exon 2 of NR3C1 is in line with transcriptional regulation of the most predominant isoform of GR reported [51]. Furthermore, Western blot analysis suggests that SOX2 positively regulates GR protein expression, where GR expression notably falls in SOX2KO cells compared to Ctl (FIG. 3A).

To understand whether SOX2 impacts GR target expression and downstream signaling, Western blotting was performed for SGK1, a canonical GR target gene [36], [37]. Despite the associated upregulation of GR expression with higher SOX2 expression observed (FIG. 3B), GR function after liganding with potent GR agonist Dexamethasone (DEX), remains robust in SOX2^{KO} cells, as demonstrated by comparable protein levels in SOX2-positive and -negative cells (FIG. 3B). Given the finding that loss of SOX2 expression was associated with robust sensitivity to ARSI, and the established role of GR in compensation for AR subsequent to ARSI, experiments were conducted to test whether effective GR pathway manipulation was affected by concurrent SOX2 expression in PC. Using the nuclear-GFP labeled CWRR1-Ctl and -SOX2^{KO} cells established earlier, proliferation was tracked using the Incucyte S3 μLive Cell Imaging platform over 150 h (˜6 days), in cells treated with: R-1881 alone (vehicle), R-1881+ENZ for AR inhibition, DEX alone to activate GR, R-1881+ENZ+DEX for AR inhibition coupled with stimulation of GR activity, or R-1881+ENZ+DEX for AR inhibition/GR activation with CORT134, a novel, selective GR modulator (SGRM) that demonstrates ability to abrogate GR signaling activity [4]. In the SOX2-positive Ctl cells, AR inhibition, as well as the AR-inhibited/GR-activated condition has no effect on their proliferative capacity compared to vehicle (FIG. 3C). The selective modulation of GR by addition of CORT134 to the AR-inhibited/GR-activated condition, also has no effect on Ctl cell proliferation (FIG. 3C). In contrast, AR-inhibited SOX2^{KO} cells had significantly lower proliferative capacity compared to vehicle (as noted earlier), and the activation of GR in AR-inhibited cells demonstrates significant recovery of proliferative capacity, compared to AR inhibition alone, p=0.0336 (FIG. 3C). Notably, the proliferation recovery observed following GR activation in AR-inhibited SOX2^{KO} cells was reversed by GR modulation with CORT134, p=0.0131 (FIG. 3C).

### CONCLUSION

Collectively, these data support the observations elsewhere that SOX2 expression drives insensitivity to nuclear hormone receptor signaling manipulation, and importantly, indicate that SOX2 loss not only sensitizes PC cells to ARSI, but to nuclear hormone receptor signaling inhibition (NHRSI) more generally, where SOX2-positive PC cells show indifference to GR agonism or antagonism with SGRM in the AR-inhibited setting.

### Example 4: WEE-1 Inhibition Potentiates the Efficacy of Nuclear Hormone Receptor Antagonism and Selective Modulation in SOX2-Positive Prostate Cancer Cells In Vitro

### Introduction

Although WEE1 acts as a tumor suppressor to regulate cell cycle control through phosphorylation and deactivation of CDK1 and CDK2 in normal tissues, WEE1 has been shown to act as an oncogene in the malignant setting, where it functions to maintain a tolerable level of genetic instability for tumor cells burdened with high endogenous replicative stress [29]. WEE1 inhibition (WEE1i) has been shown in numerous cancers to decrease disease progression [38]-[43], and WEE1i as a strategy to improve cancer patient management is currently being employed in clinical trials across several different malignancies [44]. Given the findings that, 1) WEE1 expression, along with its target CDK1, was differentially expressed both at the RNA and protein levels upon treatment with an ARSI or SGRM in SOX2^{KO} PC cells, and 2) that WEE1 expression was significantly elevated in SOX2 HI metastatic PC tissues relative to SOX2 μLOW, experiments were conducted to further investigate whether the addition of a WEE1i to NHRSIs is sufficient to reverse the aggressive phenotype of SOX2-positive PC cells.

### Method

The nuclear-GFP labeled, SOX2-positive, CWRR1-Ctl cells treated with NHRSI alone (either ENZ for AR inhibition or CORT134 for GR modulation), WEE1i's MK1775 alone, or NHRSIs+WEE1i were used to carry out the experiments.

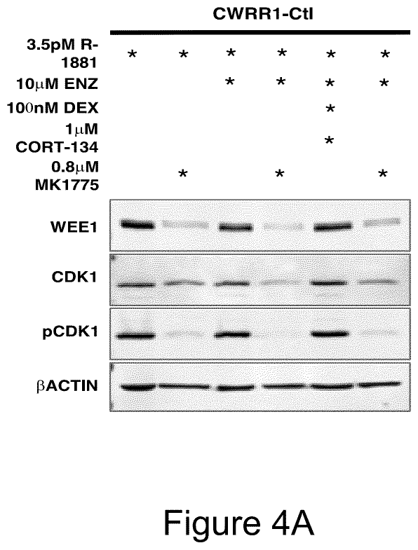

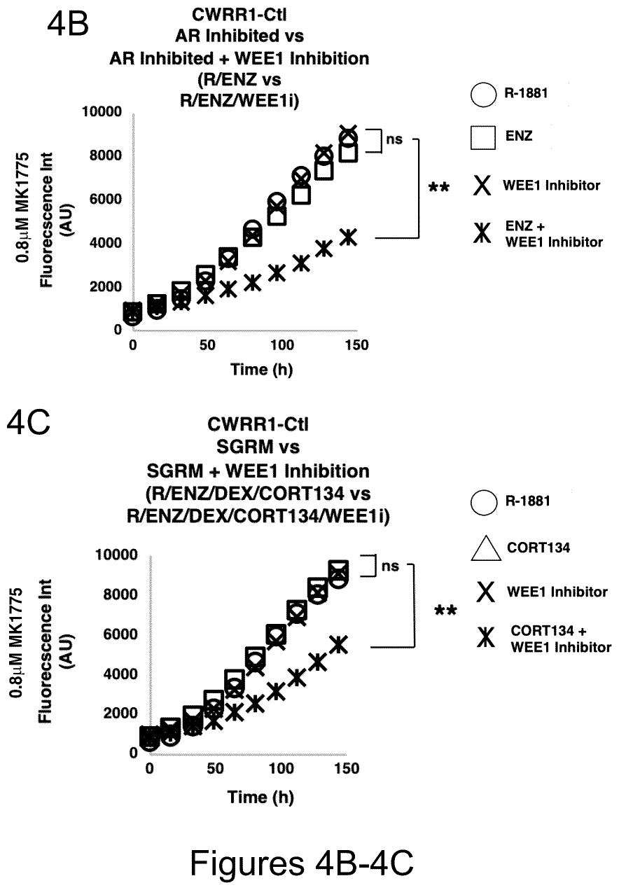

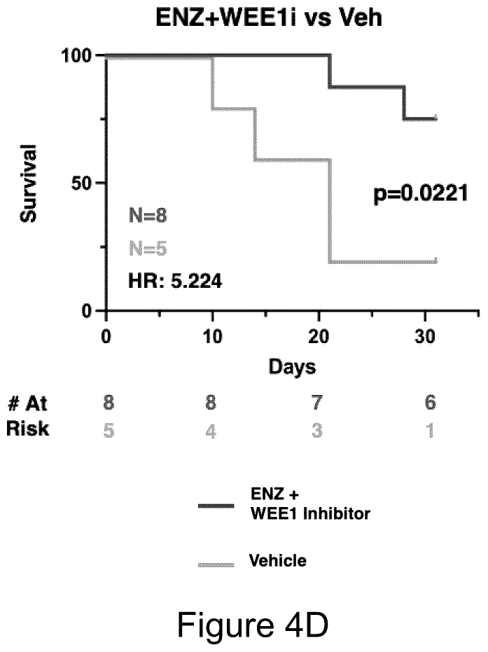

For MK1775− —treated cells, markedly lower pCDK1 protein expression was observed when WEE1i was present (FIG. 4A). As WEE1 functions to phosphorylate CDK1, decreased levels of pCDK1 are expected in WEE1i treated samples, thus demonstrating effective targeting at the respective concentrations of WEE1i's used in our studies. Notably, CDK1 protein expression was decreased in ENZ+MK1775− treated Ctl cells, and CORT-134+MK1775l − treated Ctl cells relative to MK1775 (FIG. 4A). This observation suggests that, although WEE1i treatments reach their target to phosphorylate CDK1, they are insufficient alone to drive down CDK1 expression.

It was hypothesized that a decrease in oncogenic CDK1 protein expression following concurrent WEE1i and NHRSI treatment would decrease proliferation in SOX2-positive PC cells. PC cell proliferation was measured over 150 h using the Incucyte S3 platform in each condition. Data from proliferation assays are supportive of this hypothesis, where treatment with ENZ alone has little effect on the proliferative capacity of CWRR1-Ctl cells relative to vehicle (FIG. 4B). Similarly, treatment with MK-1775 resulted in no significant difference in proliferative capacity (FIG. 4B)). However, when ENZ is combined with MK-1775, proliferation is significantly decreased (FIG. 4B; arterisk, p=0.0052). Additionally, treatment with CORT134 combined with MK-1775 significantly decreased proliferation (FIG. 4C; arterisks, p=0.0019, respectively).

To strengthen the correlation between in vitro and in silico studies thereby enhancing translational relevance, an in vivo experiment using a CRPC xenograft model was performed. Following inoculation with CWRR1-Ctl cells, tumor bearing athymic nude mice with a measured tumor volume of ˜150-200 mm³ were randomized into 4 treatment arms: Vehicle (N=5), 10 mg/kg ENZ (N=8), 50 mg/kg MK-1775 (N=8), and 10 mg/kg ENZ+50 mg/kg MK-1775 (N=8). Mice were treated daily for a period of 30 days. Endpoint for this study was defined as time to 4× tumor volume relative to volume at randomization, representing a lethal tumor burden. Mice who had not reached endpoint at 30 days of treatment were censored at day 31. There were no significant differences in time to endpoint for mice treated with ENZ or WEE1i alone relative to vehicle (not shown; p=0.1548 and p=0.0773, respectively). When mice were treated with ENZ in combination with WEE1i (FIG. 4D), a significantly longer to time to endpoint relative to vehicle-treated mice was observed (Hazard ratio 5.2 vehicle vs. combination, p=0.0221). Median time to endpoint for vehicle-, ENZ- and WEE1i-treated mice was 21, 29.5 and 29.5 days, respectively, compared to ENZ+WEE1i-treated mice, which was not reached, as 75% of mice remained without defined progression at 31 days (FIG. 4D).

### CONCLUSION

Collectively, these data strengthen the central finding that SOX2 expression renders PC cells insensitive to NHRSI, mechanistically driving resistance through altered expression of key oncogenic drivers and regulators of the cell cycle, including WEE1 and CDK1. Of note, addition of a WEE1i can efficiently prevent CDK1 phosphorylation as well as decrease its expression in vitro, and significantly slow tumor growth in vivo when combined with an ARSI. Thus, WEE1i enables re-sensitization of SOX2-positive PC cells to NHRSI treatments, reversing the aggressive SOX2-mediated PC phenotype.

### Example 5: WEE1 Inhibition, in Combination with AR Signaling Inhibition, Induces Mitotic Catastrophe in SOX2-Positive PC Cells

WEE1 inhibition functions to abrogate tumor growth by leveraging the inherent genetic instability and replicative stress caused by dysregulated cell cycle checkpoints in rapidly dividing malignant cells, ultimately driving them into mitotic catastrophe, defined as the premature or inappropriate entry of cells into mitosis, resulting in decreased proliferation and cell death mediated by various mechanisms, including apoptosis and senescence [52]. Given our findings that treatment with WEE1i prevents phosphorylation of CDK1 (FIG. 4A), decreases proliferative capacity in vitro (FIGS. 4B-4C), and slows tumor growth in vivo (FIG. 4D), we set out to investigate whether mitotic catastrophe underlies the observed therapeutic efficacy in SOX2-positive PC cells.

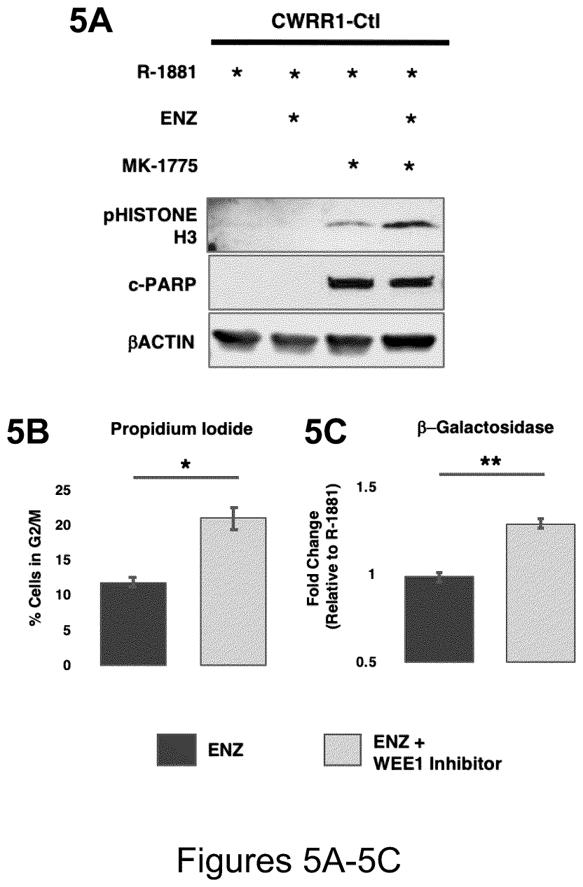

WEE1 plays a central role in negatively regulating mitotic phase entry (i.e. the G2/M checkpoint) through phosphorylation of CDK1 [29]. We hypothesized that treatment of PC cells with a WEE1i will permit premature transition through the G2/M checkpoint, leading to accumulation of cells at the G2/M phase of the cell cycle, ultimately resulting in cell death via mitotic catastrophe. As shown by Western blot, phosphorylated Histone H3 (pHistone H3), which is increased in cells at the M phase as a result of phosphorylation during chromatin condensation [53], is low in SOX2-positive CWRR1-Ctl cells when AR is agonized or antagonized but increases appreciably in cells treated with a WEE1i alone or in combination with ENZ (5A). SOX2-positive Ctl cells stained with propidium iodide, a nuclear staining dye commonly used to define cell cycle status, demonstrated significantly higher accumulation of cells at G2/M phase in ENZ+/WEE1i-treated cells (FIG. 5B) relative to ENZ alone (FIG. 5B) p=0.045. PARP-1 is a known substrate of caspase-mediated apoptotic cell death, and cleaved PARP (c-PARP) is a hallmark of apoptosis [54]. As hypothesized, WEE1i treatment leads to increased c-PARP (FIG. 5A). Detection of β-galactosidase activity, widely used as a biomarker of cellular senescence [55], and a component of mitotic catastrophe (presumably induced by WEE1i), was also assessed. β-galactosidase levels were significantly higher in ENZ+WEE1i-treated CWRR1-Ctl cells compared to treatment with ENZ alone (FIG. 5C; p=0.002).

Taken together, our data support the hypothesis that WEE1i permits dysregulated mitotic phase entry, resulting in mitotic catastrophe-mediated cell death through apoptosis (marked by cleavage of PARP) and cellular senescence (marked by β-galactosidase activity). Importantly, treatment of SOX2-positive PC cells with an NHRSI alone was not sufficient to induce this phenotype, which was only observed when an NHRSI was administered in combination with WEE1i.

### Example 6: WEE1 Inhibition, in Combination with GR Antagonism, Inhibits Proliferation of SOX2-Positive Neuroendocrine PC Cells, In Vitro

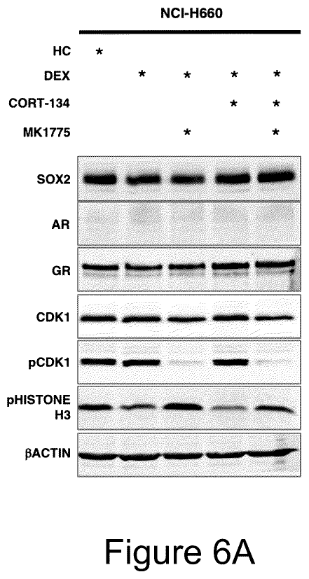

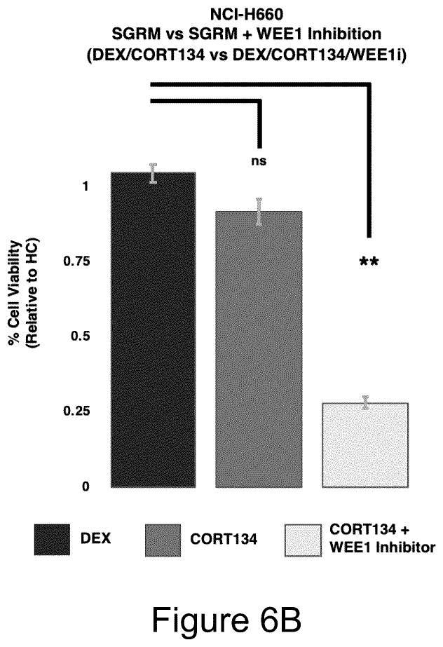

The neuroendocrine subtype is described as an aggressive variant of PC characterized by an altered genetic, transcriptional, and epigenetic program that diverges from the typical luminal phenotype found in prostate adenocarcinoma [56]. A critical consequence of the neuroendocrine phenotype in PC is the lack of AR expression, often accompanied by decreased sensitivity to ARSI [56]. Given other reports describing a role for SOX2 in driving the neuroendocrine PC phenotype [13], [56], and the SOX2-dependent NHRSI phenotype we have described herein (FIGS. 3A-3C), we sought to determine whether NCI-H660, an AR-negative neuroendocrine-derived PC cell line, would be positive for SOX2 expression and susceptible to WEE1i. Demonstrated by Western blot, NCI-H660 shows strong, consistent SOX2 protein expression across several treatment conditions (FIG. 6A). Interestingly, strong GR expression was also observed in NCI-H660 cells across all treatment conditions tested (FIG. 6A). As our previous data describes SOX2-dependent NHRSI insensitivity in AR-expressing prostate adenocarcinoma and its reversal following WEE1i (FIGS. 4A-4D), we investigated 1) whether NCI-H660 neuroendocrine PC cells are insensitive to selective GR modulation, and 2) if combining selective GR modulation with WEE1 inhibition could positively impact NHRSI efficacy. NCI-H660 cells used in this experiment were supplemented with 5% CSS and grown in RPMI-HITES media as described earlier with the following modifications: 1) R-1881 and ENZ treatments were omitted, as the neuroendocrine PC subtype characteristically has weak to absent AR expression, which obviates the need for AR agonism or antagonism, and 2) 100 nM DEX was substituted for 10 nM hydrocortisone (HC) to stay in line with previous experiments agonizing GR and to prevent overstimulation of glucocorticoid activity. We first confirmed specific WEE1i targeting in NCI-H660 cells, where markedly decreased pCDK1 protein expression was observed as well as a slight decrease in CDK1 by Western blot in WEE1i-containing treatment conditions (FIG. 6A). To investigate drug sensitivity, NCI-H660 cells were seeded onto a 96-well plate and treated with DEX (GR agonism), CORT134 alone (GR antagonism) or MK-1775+CORT134 (GR modulation/WEE1i inhibition). After 6 days, the CellTiter Blue Assay was used to measure cell viability across treatment conditions. Relative to control, DEX-treated (blue bar) and CORT134 alone-treated cells (yellow bar) showed minimal differences in cell viability (FIG. 6B). When CORT134 was given in combination with MK-1775, however, cell viability dramatically decreased relative to control, p=0.002 (FIG. 6B). Additionally, detection of pHistone H3, a marker of mitotic catastrophe, increased in MK-1775− and MK-1775+ CORT134-treated conditions relative to DEX and CORT134 treatment alone (FIG. 6A).

Our findings in NCI-H660 cells buttress our overarching hypotheses that, endogenously SOX2-expressing PC cells, whether adenocarcinoma- or neuroendocrine-derived, can similarly be re-sensitized to NHRSI by the addition of WEE1i (FIG. 7).

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

### REFERENCES CITED

[1]R. L. Siegel, K. D. Miller, N. S. Wagle, and A. Jemal, “Cancer statistics, 2023.,” CA. Cancer J. Clin., vol. 73, no. 1, pp. 17-48, 2023.
[2]R. B. Montgomery et al., “Maintenance of intratumoral androgens in metastatic prostate cancer: A mechanism for castration-resistant tumor growth,” Cancer Res., vol. 68, no. 11, pp. 4447-4454, 2008.
[3]Y. Ho and S. M. Dehm, “Androgen receptor rearrangement and splicing variants in resistance to endocrine therapies in prostate cancer,” Endocrinology, vol. 158, no. 6, pp. 1533-1542, 2017.
[4]J. Kach et al., “Selective Glucocorticoid Receptor Modulators (SGRMs) Delay Castrate-Resistant Prostate Cancer Growth,” Mol. Cancer Ther., 2017.
[5]V. K. Arora et al., “XGlucocorticoid receptor confers resistance to antiandrogens by bypassing androgen receptor blockade,” Cell, 2013.
[6]D. J. Crona and Y. E. Whang, “Androgen receptor-dependent and -independent mechanisms involved in prostate cancer therapy resistance,” Cancers (Basel)., vol. 9, no. 6, 2017.
[7]S. Kregel et al., “Sox2 is an androgen receptor-repressed gene that promotes castration-resistant prostate cancer.,” PLoS One, vol. 8, no. 1, p. e53701, January 2013.
[8]M. Piva et al., “Sox2 promotes tamoxifen resistance in breast cancer cells,” EMBO Mol. Med., vol. 6, no. 1, pp. 66-79, 2014.
[9]K. Takeda et al., “Sox2 is associated with cancer stem-like properties in colorectal cancer,” Sci. Rep., vol. 8, no. 1, pp. 1-9, 2018.
[10]W. Xiao et al., “SOX2 Promotes Brain Metastasis of Breast Cancer by Upregulating the Expression of FSCN1 and HBEGF,” Mol. Ther.—Oncolytics, vol. 17, no. June, pp. 118-129, 2020.
[11]N. Karachaliou, R. Rosell, and S. Viteri, “The role of SOX2 in small cell lung cancer, lung adenocarcinoma and squamous cell carcinoma of the lung,” Transl. Lung Cancer Res., vol. 2, no. 3, pp. 172-179, 2013.
[12]L. De Wet et al., “SOX2 mediates metabolic reprogramming of prostate cancer cells,” no. November, pp. 1-13, 2021.
[13]P. Mu et al., “SOX2 promotes lineage plasticity and antiandrogen resistance in TP53- and RB1-deficient prostate cancer,” Science (80-.)., 2017.
[14]H. Kita-Matsuo et al., “Lentiviral vectors and protocols for creation of stable hESC lines for fluorescent tracking and drug resistance selection of cardiomyocytes,” PLoS One, vol. 4, no. 4, 2009.
[15]A. M. Bolger, M. Lohse, and B. Usadel, “Trimmomatic: A flexible trimmer for Illumina sequence data,” Bioinformatics, vol. 30, no. 15, pp. 2114-2120, 2014.
[16]B. Langmead and S. L. Salzberg, “Fast gapped-read alignment with Bowtie 2,” Nat. Methods, vol. 9, no. 4, pp. 357-359, 2012.
[17]Y. Liao, G. K. Smyth, and W. Shi, “FeatureCounts: An efficient, general purpose program for assigning sequence reads to genomic features,” Bioinformatics, vol. 30, no. 7, pp. 923-930, 2014.
[18]M. I. Love, W. Huber, and S. Anders, “Moderated estimation of fold change and dispersion for RNA-seq data with DESeq2,” Genome Biol., vol. 15, no. 12, pp. 1-21, 2014.
[19]Y. Liao, J. Wang, E. J. Jaehnig, Z. Shi, and B. Zhang, “WebGestalt 2019: gene set analysis toolkit with revamped UIs and APIs,” Nucleic Acids Res., vol. 47, no. W1, pp. W199-W205, 2019.
[20]V. K. Mootha et al., “PGC-1α-responsive genes involved in oxidative phosphorylation are coordinately downregulated in human diabetes,” Nat. Genet., vol. 34, no. 3, pp. 267-273, 2003.
[21]A. Subramanian et al., “Gene set enrichment analysis: A knowledge-based approach for interpreting genome-wide expression profiles,” Proc. Natl. Acad. Sci. U.S.A, vol. 102, no. 43, pp. 15545-15550, 2005.
[22]D. Robinson et al., “Integrative clinical genomics of advanced prostate cancer,” Cell, 2015.
[23]J. Semenas, N. Dizeyi, and J. L. Persson, “Enzalutamide as a second-generation antiandrogen for treatment of advanced prostate cancer,” Drug Design, Development and Therapy. 2013.
[24]J. N. Davis et al., “Elevated E2F1 inhibits transcription of the androgen receptor in metastatic hormone-resistant prostate cancer,” Cancer Res., vol. 66, no. 24, pp. 11897-11906, 2006.
[25]D. P. Hollern et al., “E2F1 Drives Breast Cancer Metastasis by Regulating the Target Gene FGF13 and Altering Cell Migration,” Sci. Rep., vol. 9, no. 1, pp. 1-13, 2019.
[26]P. Liu, T. P. Kao, and H. Huang, “CDK1 promotes cell proliferation and survival via phosphorylation and inhibition of FOXO1 transcription factor,” Oncogene, 2008.
[27]Q. Xia, Y. Cai, R. Peng, G. Wu, Y. Shi, and W. Jiang, “The CDK1 inhibitor R03306 improves the response of BRCA-proficient breast cancer cells to PARP inhibition,” Int. J. Oncol., vol. 44, no. 3, pp. 735-744, 2014.
[28]G. T. Magnussen, R. Holm, E. Emilsen, A. K. R. Rosnes, A. Slipicevic, and V. A. Florenes, “High expression of Wee1 is associated with poor disease-free survival in Malignant Melanoma: Potential for targeted therapy,” PLoS One, vol. 7, no. 6, pp. 1-8, 2012.
[29]A. Ghelli Luserna Di Rora, C. Cerchione, G. Martinelli, and G. Simonetti, “A WEE1 family business: Regulation of mitosis, cancer progression, and therapeutic target,” J. Hematol. Oncol., vol. 13, no. 1, pp. 1-17, 2020.
[30]L. Ai et al., “The p16 (CDKN2a/INK4a) tumor-suppressor gene in head and neck squamous cell carcinoma: A promoter methylation and protein expression study in 100 cases,” Mod. Pathol., vol. 16, no. 9, pp. 944-950, 2003.
[31]K. W. Tam et al., “CDKN2A/p16 inactivation mechanisms and their relationship to smoke exposure and molecular features in non-small-cell lung cancer,” J. Thorac. Oncol., vol. 8, no. 11, pp. 1378-1388, 2013.
[32]M. H. Barcellos-Hoff and R. J. Akhurst, “Transforming growth factor-β in breast cancer: Too much, too late,” Breast Cancer Res., vol. 11, no. 1, 2009.
[33]K. Pardali and A. Moustakas, “Actions of TGF-β as tumor suppressor and pro-metastatic factor in human cancer,” Biochim. Biophys. Acta—Rev. Cancer, vol. 1775, no. 1, pp. 21-62, 2007.
[34]P. S. Larson et al., “CDKN1C/p57kip2 is a candidate tumor suppressor gene in human breast cancer,” BMC Cancer, vol. 8, pp. 1-10, 2008.
[35]P. Vlachos, U. Nyman, N. Hajji, and B. Joseph, “The cell cycle inhibitor p57Kip2 promotes cell death via the mitochondrial apoptotic pathway,” Cell Death Differ., vol. 14, no. 8, pp. 1497-1507, 2007.
[36]O. A. Itani, K. Z. Liu, K. L. Cornish, J. R. Campbell, and C. P. Thomas, “Glucocorticoids stimulate human sgk1 gene expression by activation of a GRE in its 5′-flanking region,” Am. J. Physiol.—Endocrinol. Metab., vol. 283, no. 5 46-5, pp. 971-979, 2002.
[37]M. K. Webster, L. Goya, Y. Ge, A. C. Maiyar, and G. L. Firestone, “Characterization of sgk, a novel member of the serine/threonine protein kinase gene family which is transcriptionally induced by glucocorticoids and serum,” Mol. Cell. Biol., vol. 13, no. 4, pp. 2031-2040, 1993.
[38]Y. Yin et al., “Wee1 inhibition can suppress tumor proliferation and sensitize p53 mutant colonic cancer cells to the anticancer effect of irinotecan,” Mol. Med. Rep., 2018.
[39]E. Caiola et al., “Wee1 inhibitor M1K1775 sensitizes KRAS mutated NSCLC cells to sorafenib,” Sci. Rep., 2018.
[40]M. Zhang et al., “WEE1 inhibition by MIK1775 as a single-agent therapy inhibits ovarian cancer viability,” Oncol. Lett., 2017.
[41]N. V. Rajeshkumar et al., “MK-1775, a potent Wee1 inhibitor, synergizes with gemcitabine to achieve tumor regressions, selectively in p53-deficient pancreatic cancer xenografts,” Clin. Cancer Res., 2011.
[42]D. H. Ha et al., “Antitumor effect of a WEE1 inhibitor and potentiation of olaparib sensitivity by DNA damage response modulation in triple-negative breast cancer,” Sci. Rep., 2020.
[43]A. L. Richer, J. M. Cala, K. O'Brien, V. M. Carson, L. J. Inge, and T. G. Whitsett, “WEE1 kinase inhibitor AZD1775 has preclinical efficacy in LKB1-deficient non-small cell lung cancer,” Cancer Res., 2017.
[44]A. Kong and H. Mehanna, “WEE1 Inhibitor: Clinical Development,” Curr. Oncol. Rep., vol. 23, no. 9, 2021.
[45]B. M. Ku et al., “Mutational status of TP53 defines the efficacy of Wee1 inhibitor AZD1775 in KRAS -mutant non-small cell lung cancer,” Oncotarget, vol. 8, no. 40, pp. 67526-67537, 2017.
[46]A. van Bokhoven et al., “Molecular characterization of human prostate carcinoma cell lines.,” Prostate, vol. 57, no. 3, pp. 205-25, November 2003.
[47]J. PosthumaDeBoer et al., “WEE1 inhibition sensitizes osteosarcoma to radiotherapy,” BMC Cancer, vol. 11, pp. 8-11, 2011.
[48]Y. Y. Lee et al., “Anti-Tumor Effects of Wee1 Kinase Inhibitor with Radiotherapy in Human Cervical Cancer,” Sci. Rep., vol. 9, no. 1, pp. 1-11, 2019.
[49]Z. J. Han, Y. H. Feng, B. H. Gu, Y. M. Li, and H. Chen, “The post-Translational modification, SUMOylation, and cancer (Review),” Int. J. Oncol., vol. 52, no. 4, pp. 1081-1094, 2018.
[50]S. Zhang, X. Xiong, and Y. Sun, “Functional characterization of SOX2 as an anticancer target,” Signal Transduct. Target. Ther., vol. 5, no. 1, 2020.
[51]D. Duma, C. M. Jewell, and J. A. Cidlowski, “Multiple glucocorticoid receptor isoforms and mechanisms of post-translational modification,” J Steroid Biochem Mol Biol, vol. 102, no. 1-5, pp. 11-21, 2006.
[52]H. Vakifahmetoglu, M. Olsson, and B. Zhivotovsky, “Death through a tragedy: mitotic catastrophe,” Cell Death Differ, vol. 15, pp. 1153-1162, 2008.
[53]E. Hightower, G. N. Fuller, I. E. Mccutcheon, K. R. Hess, and J. K. Devin, “Phospho-histone H3 (pHH3) immuno-reactivity as a prognostic marker in non-functioning pituitary adenomas,” Pituitary, vol. 15, no. 4, pp. 556-61, 2013.[54]G. V. Chaitanya, J. S. Alexander, and P. P. Babu, “PARP-1 cleavage fragments: signatures of cell-death proteases in neurodegeneration,” Cell Commun Signal, vol. 8, no. 31, pp. 1-11, 2010.
[55]K. Itahana et al, “Methods to detect biomarkers of cellular senescence: the senescence-associated b-galactosidase assay” Biological Aging: Methods and Protocols (Methods in Molecular Biology), vol. 1048, 2013.
[56]H. Li et al., “SOX2 has dual functions as a regulator in the progression of neuroendocrine prostate cancer,” Lab. Investig., vol. 2, pp. 570-582, 2020.
[57]N. B. Larson et al., “Comprehensively Evaluating cis-Regulatory Variation in the Human Prostate Transcriptome by Using Gene-Level Allele-Specific Expression,” Am. J. Hum. Genet., vol. 96, no. 6, pp. 869-882, 2015.
[58]H. Beltran et al., “Molecular characterization of neuroendocrine prostate cancer and identification of new drug targets,” Cancer Discov., vol. 1, no. 6, pp. 487-95, 2011.
[59]H. M. Nguyen et al., “LuCaP Prostate Cancer Patient-Derived Xenografts Reflect the Molecular Heterogeneity of Advanced Disease and Serve as Models for Evaluating Cancer Therapeutics,” Prostate, vol. 77, no. 6, pp. 654-671, 2017.
[60]D. Pflueger et al., “Discovery of non-ETS gene fusions in human prostate cancer using next-generation RNA sequencing,” Genome Res., vol. 21, no. 1, pp. 56-67, 2011.
[61]D. Robinson et al., “Integrative clinical genomics of advanced prostate cancer,” Cell, vol. 161, no. 5, pp. 1215-1228, 2015.
[62]J. R. Prensner et al., “Transcriptome sequencing across a prostate cancer cohort identifies PCAT-1, an unannotated lincRNA implicated in disease progression,” Nat. Biotechnol., vol. 29, no. 8, pp. 742-749, 2011.

Sequences

| SEQ ID | | |
|---|---|---|
| NO: | Name | Sequence |
| 1 | 5′ adapter | AGATCGGAAGAGCACACGTCTGAACTC |
| | | CAGTCA |
| 2 | 5′ adapter | AGATCGGAAGAGCGTCGTGTAGGGAAA |
| | (read 2) | GAGTGT |

1. A method of treating prostate cancer in a subject, the method comprising:
administering to the subject a therapeutically effective amount of (i) a WEE1 inhibitor or a pharmaceutically acceptable salt thereof; and (ii) a nuclear hormone receptor signaling inhibitor (NHRSI) or a pharmaceutically acceptable salt thereof.

2. The method of claim 1, wherein the prostate cancer is SOX2-positive.

3. The method of claim 1, wherein the prostate cancer is advanced prostate cancer.

4. The method of claim 1, wherein the prostate cancer is a metastatic prostate cancer.

5. The method of claim 1, wherein the prostate cancer is a castration-resistant prostate cancer.

6. The method of claim 1, wherein the prostate cancer is a neuroendocrine prostate cancer.

7. The method of claim 1, wherein the WEE1 inhibitor is MK-1775.

8. The method of claim 1, wherein the NHRSI is enzalutamide (ENZ) and/or relacorilant.

9. The method of claim 1, wherein the NHRSI is an androgen receptor signaling inhibitor and/or a selective glucocorticoid receptor-modulating inhibitor.

10. The method of claim 9, wherein the androgen receptor signaling inhibitor is MDV3100, ARN-509, flutamide, bicalutamide, nilutamide, apalutamide, enzalutamide, AZD3514, darolutamide, or cyproterone acetate.

11. The method of claim 10, wherein the androgen receptor signaling inhibitor is enzalutamide.

12. The method of claim 9, wherein the selective glucocorticoid receptor-modulating inhibitor is mifepristone or relacorilant (CORT134).

13. The method of claim 9, wherein the selective glucocorticoid receptor-modulating inhibitor is relacorilant (CORT134).

14. The method of claim 1, wherein the WEE1 inhibitor and nuclear hormone receptor signaling inhibitor are administered simultaneously or sequentially.

15. The method of claim 1, wherein the WEE1 inhibitor and/or nuclear hormone receptor signaling inhibitor are administered orally, intravenously, subcutaneously, or intratumorally.

16. A method of treating a subject having prostate cancer, the method comprising:
a) selecting a subject having prostate cancer, wherein the prostate cancer exhibits resistance to treatment with a nuclear hormone receptor signaling inhibitor; and
b) administering to the selected subject (i) one or more WEE1 inhibitors or a pharmaceutically acceptable salt thereof, and (ii) a nuclear hormone receptor signaling inhibitor (NHRSI) or a pharmaceutically acceptable salt thereof.

17. The method of claim 16, wherein the prostate cancer is SOX2-positive.

18. The method of claim 17, wherein the administering reduces tumor growth, invasiveness, progression, recurrence, and/or metastasis of the SOX2-positive prostate cancer in the subject.

19. The method of claim 17, wherein the method decreases proliferation in SOX2-positive prostate cancer cells, and re-sensitizes prostate cancer cells to treatment with an androgen receptor signaling inhibitor (ARSI) and a selective glucocorticoid receptor modulating (SGRM) therapy to nuclear hormone receptor signaling inhibition.

20. A method of treating a SOX2-positive prostate cancer in a subject, comprising:
a) administering to the subject a therapeutically effective amount of a composition comprising (i) one or more WEE1 inhibitors or a pharmaceutically acceptable salt thereof, and (ii) a nuclear hormone receptor signaling inhibitor (NHRSI) or a pharmaceutically acceptable salt thereof, wherein the composition is preferentially targeted to SOX2-positive prostate cancer cells;
b) decreasing proliferation in the SOX2-positive prostate cancer cells;
c) re-sensitizing the SOX2-positive prostate cancer to treatment with an androgen receptor signaling inhibitor (ARSI) and/or a selective glucocorticoid receptor modulating-inhibitor; and
d) reducing growth, invasiveness, progression, recurrence, and/or metastasis of the SOX2-positive prostate cancer in the subject.

21-25. (canceled)
